(19) United States
(12) Patent Application Publication
(10) Pub. No.: US 2007/0244076 A1
(43) Pub. Date: Oct. 18, 2007

(54) Site and Rate Selective Prodrug Formulations of D609 with Antioxidant and Anticancer Activity

(75) Inventors: G. Patrick Meier, Pullman, WA (US); Aiping Bai, Charleston, SC (US); Daohong Zhou, Mt. Pleasant, SC (US)

Correspondence Address: FULBRIGHT & JAWORSKI L.L.P., 600 CONGRESS AVE., SUITE 2400, AUSTIN, TX, 78701, US

(73) Assignee: MUSC Foundation Research Development, Charleston, SC (US)

(21) Appl. No.: 10/575,188

(22) PCT Filed: Oct. 8, 2004
(86) PCT No.: PCT/US04/33255

§ 371 Date: Feb. 13, 2007

Related U.S. Application Data
(60) Provisional application No. 60/509,700, filed on Oct. 8, 2003.

(51) Int. Cl. A61K 31/66; A61K 31/265; C07F 9/06

(52) U.S. Cl. 514/127; 514/512; 558/231

(57) ABSTRACT

Compounds that are heteroatom substituted alkyl derivatives of tricyclodecan-9-yl-xanthogenate, and pharmaceutical compositions of these compounds, are disclosed. Methods of treating a disease or disorder in a subject and methods of protecting normal tissues in a subject from toxicity associated ionizing radiation or chemotherapy using compositions comprising these novel compounds are also disclosed. The invention also concerns methods of treating a disease or disorder in a subject using compositions that include these novel compounds while concurrently or consecutively treating the subject with ionizing radiation or a chemotherapeutic agent.

This application claims the benefit of U.S. Provisional Application No. 60/509,700, filed Oct. 8, 2003. The entire content of the Provisional Application is incorporated by reference.

The government owns rights in the present invention pursuant to grant numbers CA 78688 and CA 86860 from the NIH and a research grant from the Department of Defense through the Hollings Cancer Center.

### BACKGROUND OF THE INVENTION

1. Field of the Invention

The present invention relates generally to the fields of organic chemistry, pharmacology, pathology, and cancer biology. More particularly, it concerns derivatives of tricyclodecan-9-yl-xanthogenate, and methods of treating a disease and methods of protecting normal tissues in a subject from toxicity associated ionizing radiation or chemotherapy.

2. Description of Related Art

Although many advances have been made in the therapy of human disease, treatments associated with significant toxicity, such as ionizing radiation (IR) and chemotherapy, are commonly used as therapeutic agents. The side effects of these forms of therapy constitute a major limitation for IR and chemotherapy, thus presenting a great challenge and opportunity to develop improved therapies with less toxicity.

Several approaches have been taken to address this challenge. One approach is to develop molecularly targeted therapies that are based on an increased understanding of the molecular mechanisms that underlie the disease process, such as neoplastic transformation in the case of cancer (Druker and Lydon, 2000; Gazdar and Minna, 2001; Gibbs, 2000). Other approaches include cytoprotectants that preferentially protect normal tissue from the toxic effects of these therapies, or sensitizers that make the diseased cells more sensitive than normal cells to IR and chemotherapy (Poggi et al., 2001; Greenberger et al., 2001). The goal of this approach is to enhance or preserve the therapeutic efficacy of IR and chemotherapy against diseased cells while minimizing their toxicity in normal tissues, thus increasing the therapeutic of these conventional modalities.

Tricyclodecan-9-yl-xanthogenate (D609) is a member of the family of compounds called xanthates (Rao, 1971). Xanthates are strong electrolytes, and readily dissociate to xanthate anions in solution. Xanthate anions and xanthic acid contain the xanthate moiety, which is a highly reductive group. Thus, D609 is a potent biological antioxidant.

Recently, it has been discovered that D609 is a potent biological antioxidant. In addition, D609 can also function as a potent cytoprotectant of normal cells from IR-induced oxidative damage (Zhou et al., 2001). Mouse splenic lymphocytes pre-treated with D609 displayed a significant reduction in IR-induced reactive oxygen species (ROS) production, and protein and lipid peroxidation. Moreover, after exposure to IR, levels of intracellular reduced glutathione (GSH) declined in untreated lymphocytes but remained steady in the cells treated with D609 (Li et al., 1998).

There is substantial evidence that D609 is a selective tumor cytotoxic agent. However, the mechanisms of action of D609 against tumor cells remain to be fully elucidated. D609 also functions as a potent chemopreventive agent, as shown in a two-stage mouse skin tumor model (Furstenberger et al., 1989). Unfortunately, D609 treatment exhibits only moderate antitumor activity in vivo. This may in part be related to poor pharmacokinetics.

Oxidative stress is a common etiology for many human diseases, including neurodegenerative diseases, diseases associated with ischemia and reperfusion injury, trauma, artiosclerosis, aging, cancer, and tissue injury caused by various DNA damaging agents, including UV radiation, ionizing radiation, and chemotherapeutic agents. Being a potent antioxidant and cytoprotectant, D609 has the potential to be used as a therapeutic agent for the treatment of these diseases. Indeed, it has been found that D609 pretreatment protected mice from ionizing radiation-induced death and dramatically reduced the infarct volume in the brains of mice subjected to cerebral ischemia and reperfusion injury in a murine stroke model (Yu et al., 2000).

Furthermore, D609 is a potent antiviral and anti-inflammatory agent. It can inhibit the activation of NF-KB and the expression/production of various inflammatory molecules and cytokines. Thus, it has been used as an experimental therapeutic agent for various viral and bacterial diseases, autoimmune diseases, and inflammatory diseases.

Although D609 poses a great therapeutic potential, its use as a therapeutic agent is very limited. For example, D609 is a potent tumor cell cytotoxic agent in vitro. However, D609 treatment exhibits only moderate antitumor activity in vivo. The disparity between the in vitro and in vivo antitumor activities of D609 may reflect its poor pharmacokinetics.

A “prodrug” is a pharmacologically inactive compound that can be converted into an active drug by metabolizing enzymes in the body, by non-metabolic reactions, or by utilizing both strategies (Smith and Clark, 1998). Prodrug modification of an active drug can be achieved by attaching a metabolically labile group that blocks the active site of the drug. This can be used to protect the pharmacore of a reactive compound, which leads to decreased metabolic inactivation and increased chemical stability of the compound. Ultimately, this can result in the improvement of the pharmacokinetics, safety, and therapeutic efficacy of the active compound. The xanthate moiety of D609 can be easily oxidized to form a disulfide bond, with subsequent loss of its biological activities (Rao, 1971; Zhou et al., 2001; Giron-Calle et al, 2002). This oxidative instability of D609 may contribute to its poor antitumor activity in vivo (Amtmann and Sauer, 1990; Sauer et al., 1990; Schick et al., 1989).

Therefore, the development of novel compounds that are prodrugs of D609, in which the xanthate moiety is protected, may lead to greater therapeutic efficacy of D609. These compounds may result in increased stability of D609, and improved pharmacokinetics and therapeutic efficacy of D609 as a therapeutic agent in the treatment of a wide range of disease processes, such as cancer and viral infection. Prodrug modification of D609 could also increase the efficacy of D609 as an antioxidant and as a cytoprotectant to protect normal healthy tissue. Thus, novel compounds that are prodrug modifications of D609 have the potential to provide dual therapeutic benefit against cancer, viral infection, and other diseases while concurrently protecting healthy tissue.

### SUMMARY OF THE INVENTION

The inventors have discovered that S-modification of D609 through a metabolically labile linkage will protect the xanthate moiety as the pharmacore of D609, resulting in increased oxidative stability, improved pharmacokinetics, and enhanced therapeutic efficacy of the drug. The metabolically labile linkage is the linkage of a heteroatom substituted alkyl moiety with the sulfhydryl moiety of D609. Examples of such heteroatom substituted alkyl moieties that have been found to protect the pharmacore of D609 include an alkoxyphosphoryl moiety and an alkoxylacyl moiety. These novel agents can be applied in new forms of treatment of diseases and conditions, such as cancer, radiation damage, and diseases associated with oxidative stress. These agents can also be used to protect normal tissue in a subject from the toxicity associated with treatment of a disease with ionizing radiation or a chemotherapeutic agent. In addition, these agents can be applied as a novel secondary therapy in the treatment of disease, such as cancer.

Certain embodiments of the present invention are generally concerned with compounds of formula (I): 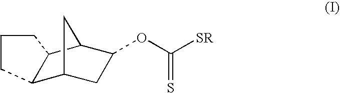
wherein R is a heteroatom substituted alkyl moiety, or a pharmaceutically acceptable salt thereof. The definition of heteroatom substituted alkyl moiety and pharmaceutically acceptable salt are discussed in detail in the specification below. The compounds of the present invention include a D609 moiety or a derivative thereof. D609 is discussed in the specification below. The compounds of the present invention include all geometrical and optical isoforms, including all geometrical and optical isoforms of D609 and variants of D609. The potassium salt of D609 is shown in FIG. 1A. The D609 moiety has three chiral centers, denoted by asterisks in FIG. 1A. As indicated by the dotted lines in the chemical structures depicted throughout this specification, the compounds of the present invention may include single enantiomers or racemic mixtures of each of the geometrical isomers and optical configurations of the claimed compound. One of ordinary skill in the art would be able to determine whether specific enantiomers have therapeutic or prophylactic activity, and would be able to synthesize a particular enantiomer.

In certain embodiments, R is an alkyl or alkoxy moiety. For example, the alkoxy moiety may be an alkoxyphosphoryl moiety or an alkoxyacyl moiety. In certain embodiments, the compound of formula (I) is: 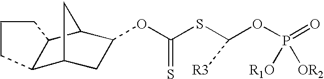
wherein R¹, R², and R³ are independently H or alkyl moieties, or a pharmaceutically acceptable salt thereof. In further embodiments, the compound of formula (I) is: 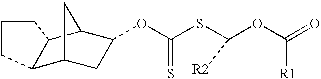
wherein R¹ and R² are independently H or alkyl moieties, or a pharmaceutically acceptable salt thereof. The structure and other features of the compounds of the present invention are discussed in greater detail in elsewhere in the specification.

The present invention also pertains to pharmaceutical compositions comprising a compound of formula (I) and a pharmaceutically acceptable excipient. Pharmaceutical compositions and pharmaceutically acceptable excipients are well-known to those of ordinary skill in the art, and are discussed in greater detail elsewhere in the specification.

Methods of treating a disease or disorder in a subject are also contemplated by the present invention, including: (1) obtaining a composition comprising a compound of formula (I): 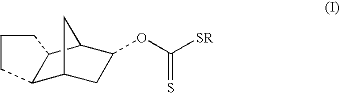
wherein R is a heteroatom substituted alkyl moiety, or a pharmaceutically acceptable salt thereof; and (2) administering a therapeutically effective amount of the composition to the subject. In certain embodiments, R is an alkyl or alkoxy moiety. For example, the alkoxy moiety may be an alkoxyphosphoryl or alkoxyacyl moiety. The examples of compounds wherein R is an alkoxyphosphoryl or alkoxyacyl moiety that were discussed above also apply to these methods.

In certain embodiments of the present methods, the subject is a mammal. For example, the mammal may be a human. The definition of disease or disorder is discussed elsewhere in the specification. Any disease or disorder is contemplated by the present invention. For example, the disease may be an autoimmune disease, and inflammatory disease, a neurodegenerative disease, a disease associated with ischemia and reperfusion injury, trauma, atherosclerosis, ageing, cancer, viral infection, UV-induced radiation injury, or ionizing radiation-induced injury. One of ordinary skill in the art would be familiar with the diseases that fall within each of these categories.

For example, the autoimmune disease may be systemic lupus, chronic thyroiditis, Graves disease, autoimmune gastritis, autoimmune hemolytic anemia, autoimmune neutropenia, or thrombocytopenia. The inflammatory disease may be rheumatoid arthritis, organ transplant rejection, graft versus host disease, endotoxemia, sepsis, septic shock, uveitis, inflammatory peritonitis, or pancreatitis. The neurodegenerative disease may be Alzheimer disease, Parkinson's disease, Huntington's disease, Kennedy's disease, prion disease, multiple sclerosis, amyotrophic lateral sclerosis, or spinal muscular atrophy. The disease associated with ischemia and reperfusion injury may be a stroke or myocardial infarction.

The methods of the present invention can also be applied in the treatment of cancer. Any type of cancer is contemplated for treatment by the methods of the present invention. One of ordinary skill in the art would be familiar with the many types of cancers that are known which would be amenable to treatment. For example, the cancer may be breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, leukemia, stomach cancer, pancreatic cancer, testicular cancer lymphoma, or multiple myeloma. The cancer may be localized cancer, locally invasive cancer, or metastatic disease.

Tissue destruction associated with trauma is also contemplated for treatment by the present methods. Any type of traumatic tissue damage is contemplated for treatment by the methods of the present invention. For example, the trauma may be traumatic brain injury, spinal cord injury, or burn injury. In addition, the disease or disorder may be a condition that is associated with oxidative stress. Any condition associated with oxidative stress is contemplated for treatment.

In some embodiments, the methods of the present invention also include methods of targeting delivery of a therapeutic amount of the composition to a site of disease in a subject by release of the active agent at the site of disease following administration. The active agent is released by phosphatase, esterase, or amidase activity at the site of disease following administration. The released active agent then scavenges reactive species (including oxygen and nitrogen radicals), inhibits phosphatidylcholine-specific phospholipase C (PC-PLC) and enzymes involved in sphingolipid metabolism (such as sphingomyelin synthase and various ceramidases), suppresses NF-κB activity, and decreases the production of various inflammatory molecules and cytokines.

Administration of the compositions can be by any method known to those of ordinary skill in the art. For example, the therapeutic amount of the composition can be administered by oral administration, intravenous administration, intraarterial administration, topical administration, intratumoral administration, regional administration, intrathecal administration, intraperitoneal administration, intraocular administration, or inhalational administration.

The present invention also concerns methods of protecting normal tissue in a subject from the toxicity associated with treatment of a disease with ionizing radiation or a chemotherapeutic agent, including: (1) obtaining a compound of formula (I) as discussed above, and (2) concurrently or consecutively administering to the subject a prophylactically effective amount of the composition and the ionizing radiation or chemotherapeutic agent. The structural features of formula (I) discussed above also apply to this section. For example, R can be an alkyl or alkoxy moiety. The alkoxy moiety may include, for example, an alkoxyphosphoryl or alkoxyacyl moiety. The examples of compounds previously disclosed also apply to these particular methods.

As noted above, the subject may be a mammal, such as a human. The toxicity may be associated with treatment of any disease, such as an autoimmune disease, and inflammatory disease, a neurodegenerative disease, a disease associated with ischemia and reperfusion injury, trauma, atherosclerosis, ageing, cancer, or a viral infection. Examples of these diseases discussed above also apply to these particular methods.

The definition of chemotherapeutic agent is detailed in the specification below. In certain embodiments, the chemotherapeutic agent is doxorubicin, daunorubicin, methotrexate, tamoxifen, paclitaxel, topotecan, LHRH, mitomycin C, etoposide tomudex, podophyllotoxin, mitoxantrone, colchicine, endostatin, fludarabin, mitomycin, actinomycin D, bleomycin, cisplatin, VP16, an enedyine, taxol, vincristine, vinblastine, carmustine, melphalan, cyclophosphamide, chlorambucil, busulfan, lomustine, 5-fluorouracil, gemcitabine, BCNU, or camptothecin.

Administering a prophylactically effective amount of the composition includes any route or method of administration. Examples include oral administration, intravenous administration, intraarterial administration, topical administration, local administration into a tumor, intrathecal administration, intraperitoneal administration, intraocular administration, or inhalational administration. One of ordinary skill in the art would be familiar with the range of methods of administering a composition that are available. The methods of protecting normal tissue in a subject can also concurrently include targeting delivery of a therapeutic amount of the composition to a site of disease in the subject, as discussed above in relation to therapeutic methods involving the claimed compositions.

A prophylactically effective amount of the composition is an amount that is expected to prevent the development of a particular disease or condition in a subject. The prophylactically effective amount of the composition may be administered concurrently or consecutively with the ionizing radiation or chemotherapeutic agent. The definitions of concurrent and consecutive administration are discussed in detail elsewhere in the specification, and apply to this section. In certain embodiments, the prophylactically effective amount of the composition and the ionizing radiation or chemotherapeutic agent are concurrently administered. In other embodiments, the prophylactically effective amount of the composition and the ionizing radiation or chemotherapeutic agent are consecutively administered. One of ordinary skill in the art would be familiar with techniques to determine the amount of a dose to be administered such that it is prophylactically effective.

Further embodiments of the present invention pertain to methods of treating a disease or disorder in a subject, including: (1) obtaining a composition that includes a compound of formula (I) disclosed above; and (2) concurrently or consecutively administering a therapeutically effective amount of the composition and ionizing radiation or a chemotherapeutic agent to the subject. The compounds of formula (I) disclosed in previous parts of this summary also apply to these methods. For example, in some embodiments, R in formula (I) is an alkyl or alkoxy moiety, such as an alkoxyphosphoryl moiety or an alkoxyacyl moiety.

These methods can be applied to any subject. In certain embodiments, the subject is a mammal. More particularly, the subject may be a human subject. Treatment of any disease or disorder is contemplated by the present invention. Examples of these diseases disclosed in reference to other methods in this summary also apply to these particular methods. For example, the disease may be a cancer, such as breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, leukemia, stomach cancer, pancreatic cancer, testicular cancer lymphoma, or multiple myeloma.

As noted above, any method of administration of the therapeutically effective amount of the composition is contemplated by the present invention. Examples of these methods have been previously discussed, and are also discussed elsewhere in this specification. In certain embodiments, the therapeutically effective amount of the composition and the ionizing radiation or chemotherapeutic agent are concurrently administered. In still other embodiments, the therapeutically effective amount of the composition and the ionizing radiation or chemotherapeutic agent are consecutively administered. Concurrent and consecutive administration have been previously discussed.

One of ordinary skill in the art would be able to determine the amount of the composition to be administered such that a therapeutic effect is achieved. A therapeutically effective amount of the composition is an amount that is expected to prevent progression or result in improvement in the disease or disorder, or to otherwise achieve a desired therapeutic effect.

As used herein the specification, “a” or “an” may mean one or more. As used herein in the claim(s), when used in conjunction with the word “comprising”, the words “a” or “an” may mean one or more than one. As used herein “another” may mean at least a second or more.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

It is contemplated that any embodiment discussed in this specification can be implemented with respect to any method or composition of the invention, and vice versa. Furthermore, compositions of the invention can be used to achieve methods of the invention.

The term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A, FIG. 1B, FIG. 1C: FIG. 1A chemical structure of the potassium salt of one of the isomers of D609; FIG. 1B chemical structure of one isomer of S-(Alkoxyphosphoryl) D609;

FIG. 1C chemical structure of one isomer of S-(Alkoxyacyl) D609.

FIG. 2: Scheme of the synthesis of D609 prodrugs 1, 2 and 3.

FIG. 3A, FIG. 3B, FIG. 3C: FIG. 3A structure of S-methyleneoxyacetyl D609 (prodrug 1); FIG. 16B structure of S-methyleneoxybutyryl D609 (prodrug 2); FIG. 16C structure of S-methyleneoxypivalyl D609 (prodrug 3).

FIG. 4: The synthesis scheme for the alkoxyphosphoryl prodrug S-(methyleneoxy)-D609, di(t-butoxy)phosphoryl designated as compound 7.

FIG. 5: Cyclic voltammetry of D609.

FIG. 6A, FIG. 6B: D609 protects mice from IR-induced lethality.

FIG. 7A, FIG. 7B: D609 selectively induces tumor cell death by apoptosis.

FIG. 8A, FIG. 8B, FIG. 8C: D609 enhances mouse splenic lymphocyte mitogenic responses and IFNγ production.

FIG. 9A, FIG. 9B, FIG. 9C: Comparison of the effects of D609, cyclohexyl xanthate, and tricyclodecanol on PC-PCLbc, SMS and U937 cell viability.

FIG. 10A, FIG. 10B, FIG. 10C, FIG. 10D: D609 inhibits cellular SMS activity and induces changes in the cellular levels of ceramide and DAG in U937 cells.

FIG. 11: Ceramide and H7 synergistically induce U937 cell apoptosis.

FIG. 12: PMA attenuates D609-induced U937 cell apoptosis.

FIG. 13A, FIG. 13B: Effects of D609 and/or IR on A20 cell viability and growth in vitro.

FIG. 14A, FIG. 14B: Lack of significant therapeutic effects of D609 and/or IR on A20 lymphoma in vivo.

FIG. 15: Structure and main metabolic pathway of D609.

FIG. 16A, FIG. 16B: Comparison of the stability of D609 and its prodrugs in saline. FIG. 16A D609 was dissolved in 5% methanol/saline (300 μM) at room temperature and changes in the concentration as a function of time were determined by HPLC analysis (mobile phase: 100% methanol; Retention time: 0.95±0.01 min). The data are presented as area under curves (AUC). FIG. 16B D609 prodrugs 1, 2 and 3 were dissolved in 5% methanol/saline (300 μM) at room temperature and changes in the concentration as a function of time were determined by HPLC analysis (mobile phase: 100% methanol; Retention time: 1, 1.77±0.03 min; 2 1.97±0.01 min; and 3 2.08±0.01). The AUCs were converted to concentrations (μM) from a linear standard curve constructed for each of these prodrugs by HPLC.

FIG. 17A, FIG. 17B: Proposed hydrolytic path of D609 prodrugs by esterase or alkaline phosphatase.

FIG. 18A, FIG. 18B, FIG. 18C: Esterase-catalyzed hydrolysis of D609 prodrugs. Prodrugs (300 μM in 15% DMSO/PBS, pH 7.4), 1 (FIG. 18A), 2 (FIG. 18B) and 3 (FIG. 18C), were incubated at 37° C. in the presence of 0.1 unit/ml PLE. Hydrolysis of D609 prodrugs was monitored at various time points by HPLC analysis and the release of D609 was determined by measuring the calorimetric reaction of D609 with DTNB (300 μM). The data are presented as the mean±SEM of three independent assays.

FIG. 19: Hydrolysis of D609 prodrug in plasma. Prodrug 2 was incubated in rat plasma (300 μM in 15% DMSO/plasma) at 37° C. At various times of the incubation, the hydrolysis of prodrug 2 was monitored by HPLC analysis, and the release of D609 was determined by measuring the colorimetric reaction of D609 with DTNB (3 mM). The data are presented as mean±SE of three independent assays.

FIG. 20A, FIG. 20B: Prodrug modification increases D609 tumor cell cytotoxicity. FIG. 20A Effects of prodrug 2 and D609 on U937 cell viability. U937 cells (5×10⁵/ml) were incubated in 96-well plates for 48 h with several concentrations of prodrug 2 or D609. No exogenous esterase was added as FBS contains sufficient esterases (about 1 unit/ml) to hydrolyze the prodrug. Cell viability was analyzed by MTT assay. The results are expressed as a percentage relative to control untreated cells and presented as means±SEM of triplicates. A representative assay of three independent assays is shown. FIG. 20B Prodrug 2 and D609 induce apoptosis in U937 cells. U937 cells (5×10⁵/ml) were incubated with vehicle (0.5% DMSO) or 177 μM D609 or prodrug 2 in vehicle. Apoptotic cell death was analyzed by determination of the sub G_{0/1} cells using a flow cytometer. Representative flow cytometric analyses are shown.

FIG. 21: Prodrug modification increases the inhibitory effect of D609 on sphingomyelin synthase (SMS). Cell lysates were prepared from U937 cells incubated with 177 μM D609 or prodrug 2 for 0.5, 1 and 2 h. NBD-C₆-ceramide and PC were incubated with the cell lysates containing 50 μg proteins for 30 min at 30° C. The formation of NBD-C₆-sphingomyelin was analyzed by TLC and quantified by determination of the fluorescent intensity using a phosphorimager. The SMS activity is expressed as % of control cells incubated with vehicle (0.5% DMSO). The results are presented as means±SEM (n=3). *p<0.05.

### DESCRIPTION OF ILLUSTRATIVE EMBODIMENTS

The present invention seeks to exploit the inventors' discovery by providing for novel compounds that are heteroatom substituted alkyl derivatives of tricyclodecan-9-yl-xanthogenate (D609). D609 was originally developed as an antitumor and antiviral agent. These novel compounds have increased oxidative stability and are expected to have improved pharmacokinetics and enhanced therapeutic efficacy compared to D609.

These novel compounds can be applied as novel therapeutic agents in the treatment of a wide range of diseases, including autoimmune diseases, inflammatory diseases, neurodegenerative diseases, diseases associated with ischemia and reperfusion injury, trauma, atherosclerosis, ageing, cancer, viral infection, and UV and ionizing radiation-induced injury and tissue damage. In addition, these compounds not only have enhanced therapeutic efficacy compared to D609, but they also can protect normal tissue and are potent antioxidants.

### A. Tricyclodecan-9-yl-xanthogenate (D609) and Prodrugs of D609

1. D609

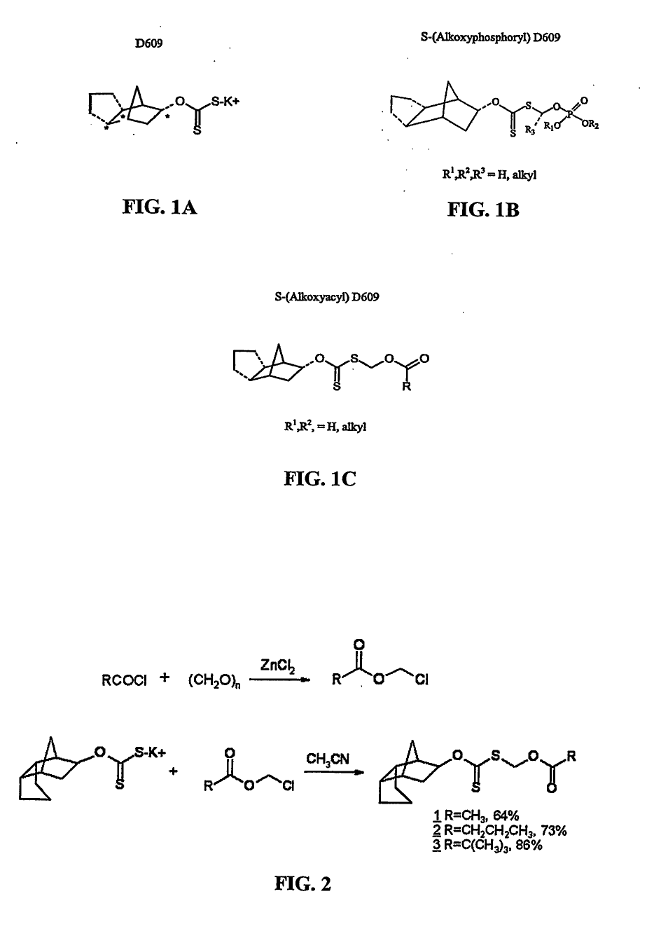

As used herein, tricyclodecan-9-yl-xanthogenate, or D609, refers not only to the xanthate derivative of D609, but also refers to any xanthate anion of D609, which may include a cation of an alkali metal. Any cation of an alkali metal is contemplated for inclusion in the definition of D609. Although the compound known as D609 is the C-endo, O-exo isomer, the claimed compounds of the present invention are herein more broadly defined to include any and all geometrical and optical isomers of D609 and variants of D609. FIG. 1A depicts one example of D609, which is a potassium salt of D609. The three chiral centers are designated by asterisks in FIG. 1A. As indicated by the dotted lines in the structures depicted throughout this specification, the compounds of the present invention may include single enantiomers or racemic mixtures of each of the geometrical isomers and optical configurations of the claimed compounds. The compounds of the claimed invention include any and all optical and geometrical isomers. D609 is a member of the family of compounds called xanthates, which are formed by the reaction of carbon disulfide, an alcohol, and an alkali in an equal stoichiometric ratio with elimination of water. Xanthates have the general structure: ROCS₂M, where R stands for an alkyl hydrocarbon moiety and M denotes a monovalent cation such as potassium. Xanthates are strong electrolytes, and readily dissociate to xanthate anions (and cations of alkali metals) in solution.

Upon reacting with an oxidant, xanthates are oxidized to dixanthogens, which contain a disulfide bond (Rao, 1971), which implies that D609 and other xanthate derivatives can function as potent biological antioxidants. This assumption is supported by the finding that D609 effectively inhibits hydroxyl radical-mediated oxidation of dihydrorhodamine 123 (DHR) in a dose-dependent manner (Zhou et al., 2001). In addition, D609 inhibits the formation of the α-phenyl-tert-butylnitrone (PBN)-free radical spin adducts and lipid peroxidation of synaptosomal membranes by hydroxyl radical (Zhou et al., 2001).

GSH is one of the major intracellular defense molecules against oxidative stress and also has been shown to play an important role in radiation protection (Hospers et al., 1999). Maintenance of a steady level of intracellular GSH by D609 may contribute to the suppression of IR-induced oxidative damage. In addition, D609 could protect normal cells from IR-induced damage by inhibiting phosphatidylcholine-specific phospholipase C(PC-PLC). By inhibiting PC-PLC, D609 could reduce the production of diacylglycerol (DAG) that is coupled to the activation of acidic sphingomyelinase (aSMase) and the subsequent production of ceramide via hydrolysis of sphingomyelin by aSMase, and thus, protect normal cells, particularly endothelial cells, from IR-induced apoptosis (Schutze et al., 1991; Schutze et al., 1992; Paris et al., 2001; Santana et al., 1996).

Importantly, D609 does not protect tumor cells from IR-induced cell death, nor does it protect tumor cells from chemotherapeutic agent-induced apoptosis (Bettaieb et al., 1999). These findings indicate that D609 has the ability to selectively protect normal cells but not tumor cells from IR- and chemotherapeutic agent-induced cytotoxicity. The mechanisms that underlie the difference between normal cells and tumor cells in their response to D609-mediated cytoprotection have yet to be determined.

There is substantial evidence that D609 is a selective tumor cytotoxic agent. The list of transformed and malignant cell types that are sensitive to D609 toxicity is expanding and now includes bovine papilloma virus type 1 (BPV-1)- and SV40-transformed animal and human fibroblasts, various leukemia/lymphoma cells and different solid tumor cells with only a few exceptions (Amtmann and Sauer, 1987; Porn-Ares et al., 1997; Schick et al., 1989; Enomoto et al., 2000). Even some drug-resistant tumor cells, such as methotrexate- and adriamycin-resistant L1210 and S180 cells, are susceptible to D609 cytotoxicity (Schick et al., 1989).

In contrast, under the same in vitro cell culture conditions, D609 did not show any cytotoxicity against normal human fibroblasts or peripheral blood lymphocytes (Amtmann and Sauer, 1987). In fact, D609 enhances mitogen-stimulated mouse splenic lymphocyte proliferation and cytokine production. These observations suggest that unlike other known chemotherapeutic agents that usually inhibit tumor cell growth and induce tumor cell death nonspecifically by inhibiting DNA replication or inducing DNA damage, the antitumor effect of D609 is likely the result of inhibition of a tumor-specific target.

However, the mechanisms of action of D609 against tumor cells remain to be fully elucidated. Originally, it was suggested that D609 functions as a specific inhibitor of the phosphatidylcholine-specific phospholipase C(PC-PLC), mainly based on in vitro cell-free studies using the bacterial enzyme (Amtmann, 1996; Schutze et al., 1992). PC-PLC utilizes phosphatidylcholine (PC) as substrate and hydrolyzes PC to produce diacylglycerol (DAG) and phosphocholine (PhoCho) (Schutze et al., 1992; Machleidt et al., 1996; Schutze et al., 1991). Recently, it was reported that D609 also inhibits SMS which transfers the PhoCho group from PC to ceramide and produces DAG and sphingomyelin (SM) (Lubert and Hannun, 1998; Luberto et al., 2000). These observations raise the possibility that SMS may account for some of the cellular effects that had been attributed to PC-PLC (Luberto and Hannun, 1998; Luberto et al., 2000), because both enzymes utilize PC as substrate and produce DAG as one of their products.

D609 also functions as a potent chemopreventive agent, as shown in a two-stage mouse skin tumor model (Furstenberger et al., 1989). Unfortunately, D609 treatment exhibits only moderate antitumor activity in vivo. This may in part be related to poor pharmacokinetics. As a xanthate derivative, D609 is relatively unstable in solution and in biological systems (Rao, 1971; Zhou et al., 2001; Giron-Calle et al., 2002). D609 can also be readily oxidized, resulting in loss of the xanthate moiety. Since the xanthate moiety functions as the pharmacore of D609 for many of its biological activities, a sufficient amount of D609 probably cannot reach the target tissue in vivo after administration. This oxidative instability of D609 may contribute to its poor antitumor activity in vivo (Amtmann and Sauer, 1990; Sauer et al., 1990; Schick et al., 1989).

2. Prodrugs

The compounds of the present invention are the prodrugs of D609. As depicted by the asterisks in FIG. 1A, the structure of D609 includes three chiral centers. As indicated by the dotted lines in the chemical structures depicted throughout this specification, the compounds of the present invention may include single enantiomers or racemic mixtures of each of the geometrical isomers and optical configurations of the claimed compounds. One of ordinary skill in the art would be able to determine whether specific enantiomers have therapeutic or prophylactic activity, and would be able to synthesize a particular enantiomer. Examples of structures of compounds of the present invention include S-(alkoxyphosphoryl) D609 (FIG. 1B) and S-(alkoxyacyl) D609 (FIG. 1C) compounds.

The definition of D609 is defined herein to include all isomers of D609. As the compounds of the present invention include a D609 moiety, it holds true that the compounds of the present invention therefore include D609 moieties that are of a single isoform or different isoforms. The compounds of the claimed invention include any and all optical and geometrical isomers.

It is possible that particular isoforms of the compounds of the present invention have enhanced therapeutic activity compared to other isoforms. One of ordinary skill in the art would be able to synthesize enantiomers of the compounds, and determine which isoforms have therapeutic activity.

Any method known to those of ordinary skill in the art can be used to synthesize D609 for use in synthesis of the compounds of the present invention. For example, one well-known method to synthesize D609 is described by Rao, 1971, which is herein specifically incorporated by reference. One of ordinary skill in the art would be familiar with other methods that can be used to synthesize D609.

Similarly, any method known to those of ordinary skill in the art can be used to synthesize the compounds of the present invention, which include the D609 moiety. Particular methods of synthesis of compounds of the present invention are discussed in the examples below.

Recently, the alkoxyphosphoryl group has been developed as a prodrug modification of a carboxylic acid and amine as a means for increasing the water solubility of lipophilic drugs (Nudelman et al., 2001; Krise et al., 1999a, Krise et al., 1999b). This prodrug moiety was designed to release the active drugs via a two-step process. Alkaline phosphatase catalyzes the hydrolysis of the phosphate ester and the resulting hydroxymethyl ammonium salt rapidly reverts to formaldehyde and the amine.

3. Substituents

Certain embodiments of the present invention pertain to chemical formulas that include an R group moiety (see, e.g., formulas disclosed in claims 1, 7, 23, and 37), wherein R is a heteroatom substituted alkyl moiety. A heteroatom substituted alkyl moiety is a carbon chain of one or more carbons in which the linking carbon is bonded to an atom other than carbon or hydrogen in addition to the linking group functionality. In some embodiments, the heteroatom substituted alkyl moiety may further be defined as an alkane, an alkene, an alkyne, a diene, an arene, an alkyl halide, an alkenyl halide, or an aryl halide. In certain embodiments, R is an alkoxy moiety. The alkyl or alkoxy moiety can include any number of carbon atoms. For example, in some embodiments, the alkyl or alkoxy moiety includes 1, 2, 3, 4, 5, 6, 7, 8, 9, 10, 11, 12, 13, 14, 15, 16, 17, 18, 19, 20, 22, 24, 26, 28, 30, 35, 40, or more carbon atoms. In certain embodiments, the alkoxy moiety is an alkoxyphosphoryl moiety or an alkoxyacyl moiety.

Certain embodiments of the present invention pertain to compounds that have chemical formulas wherein R is an alkoxyphosphoryl moiety or alkoxyacyl moiety that further includes a ligand designated R₁, R₂, or R₃. In these embodiments, R₁, R₂, or R₃ are independently H, alkyl, alkenyl, aryl moieties or pharmaceutically acceptable salts. Any type of alkyl moiety is contemplated for inclusion in the present invention. One of ordinary skill in the art would be familiar with the wide range of types of alkyl moieties that can be included in the compounds of the present invention.

For example, the alkyl moiety may be further defined as an alkane, an alkene, an alkyne, a diene, an arene, an alkyl halide, an alkenyl halide, or an aryl halide. The alkyl moiety may or may not be substituted. The present invention contemplates any type of substitution of the alkyl moiety. As discussed above, a compound of the present invention may comprise, but is not limited to, a diastereomer, an enantiomer, or a racemic mixture of stereoisomers.

4. Pharmaceutically Acceptable Salts

In certain embodiments, the compounds of the present invention pertain to pharmaceutically acceptable salts.

Non-toxic esters and salts which are generally prepared by reacting the free base with a suitable organic or inorganic acid are suitable for pharmaceutical use. Representative salts and esters include the following: Acetate, Lactobionate, Benzenesulfonate, Laurate, Benzoate, Malate, Bicarbonate Maleate, Bisulfate Mandelate, Bitartrate, Mesylate, Borate, Methylbromide, Bromide, Methylnitrate, Calcium Edetate, Methylsulfate, Camsylate, Mucate, Carbonate, Napsylate, Chloride, Nitrate, Clavulanate, N-methylglucamine, Citrate, ammonium salt, Dihydrochloride, Oleate, Edetate, Oxalate, Edisylate, Pamoate (Embonate), Estolate, Palmitate, Esylate, Pantothenate, Fumarate, Phosphate/diphosphate, Gluceptate, Polygalacturonate, Gluconate, Salicylate, Glutamate, Stearate, Glycollylarsanilate, Sulfate, Hexylresorcinate, Subacetate, Hydrabamine, Succinate, Hydrobromide, Tannate, Hydrochloride, Tartrate, Hydroxynaphthoate, Teoclate, Iodide, Tosylate, Isothionate, Triethiodide, Lactate, or Valerate. One of ordinary skill in the art would be familiar with these and other pharmaceutically acceptable salts that are contemplated by this invention.

The definition of “pharmaceutical” and “pharmaceutically acceptable” is defined below in this specification, and these definitions apply to this section of the specification.

### B. Diseases and Disorders to be Treated

Treatment of any disease or disorder is contemplated by the methods of treatment of the present invention. “Treating” and “treatment” are broadly defined and includes for example a slowing or halting of the progression of a disease or disorder. For example, inhibiting the growth of a lesion, such as a tumor, can also include a reduction in the size of a lesion or induction of apoptosis of the cells of the lesion. One of ordinary skill in the art would be familiar with the slowing or halting of the progression of a disease or disorder.

As used herein, “disease” refers to a pathological condition of a body part, an organ, or a system resulting from various causes, such as infection, genetic defect, or environmental stress, or any other cause, and characterized by an identifiable group of signs or symptoms. As used herein, “disorder” refers to any disturbance or derangement that affects the function of the mind or body, or any disturbance of normal physical health. An example of a disorder would be aging. A disorder may or may not be associated with a group of signs and symptoms.

Examples of diseases and disorders that are contemplated for treatment include, but are not limited to, autoimmune diseases. Examples of autoimmune diseases include systemic lupus, chronic thyroiditis, Graves disease, autoimmune gastritis, autoimmune hemolytic anemia, autoimmune neutropenia, and thrombocytopenia. Inflammatory diseases are also contemplated for treatment. Examples of inflammatory diseases include rheumatoid arthritis, organ transplant rejection, graft versus host disease, endotoxemia, sepsis, septic shock, uveitis, inflammatory peritonitis, and pancreatitis. Neurodegenerative diseases, such as Alzheimer disease, Parkinson's disease, Huntington's disease, Kennedy disease, prion disease, multiple sclerosis, amyotrophic lateral sclerosis, and spinal muscular atrophy, are also contemplated for treatment. Diseases associated with ischemia and reperfusion injury, such as stroke and heart attack, are also contemplated for treatment by the methods of the present invention. Other diseases that are contemplated for treatment include trauma, such as traumatic brain injury, spinal cord injury, and burn. Additional diseases and disorders that are contemplated for treatment by the methods of the present invention include atherosclerosis, aging, cancer, viral infection, and UV and ionizing-induced injury and tissue damage.

The cancer may be of any type of cancer, such as breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, leukemia, stomach cancer, pancreatic cancer, testicular cancer lymphoma, or multiple myeloma. The cancer may be localized cancer, locally invasive cancer, or metastatic disease.

Other diseases and disorders contemplated for treatment include any disease or disorder associated with oxidative stress, including neurodegenerative diseases, diseases associated with ischemia and reperfusion injury, trauma, arteriosclerosis, aging, cancer, and tissue injury caused by therapeutic agents, such as UV radiation, ionizing radiation, and chemotherapy.

### C. Pharmaceutical Formulations

1. Overview

The present invention contemplates pharmaceutical compositions comprising compounds of the present invention in a pharmaceutically acceptable excipient. It also contemplates methods of treating a disease or disorder in a subject that include administering a therapeutically effective amount of a composition of the present invention to a subject. In addition, the present invention pertains to methods of protecting normal tissue in a subject from the toxicity associated with treatment of a disease with ionizing radiation or a chemotherapeutic agent, which include concurrently or consecutively administering to the subject a prophylactically effective amount of the composition and the ionizing radiation or chemotherapeutic agent. The present invention also pertains to methods of treating a disease or disorder in a subject that include concurrently or consecutively administering to the subject a therapeutically effective amount of the composition and ionizing radiation or a chemotherapeutic agent to the subject.

2. Administration

a. Routes of Administration

In the context of the claimed invention, “administering” is defined to include administration of the composition by any method known to those of ordinary skill in the art. Examples of routes of administration are further discussed in the Summary of the Invention. One of ordinary skill in the art would be familiar with the wide range of routes of administration of a therapeutic composition that are available.

b. Concurrent Administration

Certain embodiments of the present invention pertain to methods that involve concurrent or consecutive administration of a prophylactically effective amount of the composition and ionizing radiation or a chemotherapeutic agent to protect normal tissue in a subject from toxicity. Other embodiments of the present invention pertain to methods involving concurrent or consecutive administration of a therapeutically effective amount of the composition and ionizing radiation or a chemotherapeutic agent to a subject for treatment of a disease or disorder.

As used herein, “concurrent” is defined to mean initiation of administration of the therapeutic or prophylactic composition at about the same time as the ionizing radiation or chemotherapeutic agent. For example, in certain embodiments of the present invention, the administration of the therapeutically or prophylactically effective amount of the composition will begin at the same time, within about 1 minute, within about 2 minutes, within about 3 minutes, within about 4 minutes, within about 5 minutes, within about 6 minutes, within about 7 minutes, within about 8 minutes, within about 9 minutes, within about 10 minutes, within about 12 minutes, within about 14 minutes, within about 16 minutes, within about 18 minutes within about 20 minutes, within about 25 minutes, within about 30 minutes, within about 35 minutes, within about 40 minutes, within about 45 minutes, within about 50 minutes, within about 55 minutes, or within about 60 minutes of beginning or ending a single dose of radiation or a chemotherapeutic agent, or any intermediate time within these intervals.

Administration of a chemotherapeutic agent is discussed further below. One of ordinary skill in the art would be familiar with administration of a chemotherapeutic agent. Some agents are administered in a single dose over a specific time interval, such as 20 minutes, 40 minutes, or over 1 hour. Other agents may be administered over a shorter interval, such as 5 minutes. One of ordinary skill in the art would be familiar with methods of administration of these agents, and time intervals over which these agents must be administered. Thus, concurrent administration of the therapeutic or prophylactic amount of the composition with the therapeutic administration may, in certain embodiments, involve beginning administration of the therapeutic agent during any time point while a single dose of the chemotherapeutic agent is being administered.

One of ordinary skill in the art would also understand that a course of a particular chemotherapeutic agent often involves administration of multiple doses of the chemotherapeutic agent over a period of time. For example, a course of a particular chemotherapeutic agent may involve administration of 3 doses of the agent over a period of three days, 1 week, or 2 weeks. One of ordinary skill in the art would be familiar with these regimens for administration of courses of different chemotherapeutic agents. Concurrent administration is thus also defined to include administration of a therapeutic or prophylactic amount of the composition at any point during the beginning and ending of a multiple dose course of therapy with a particular chemotherapeutic agent. For example, concurrent administration may include initiating administration of a course of a therapeutically effective amount of the composition on day 13 of a 15 day course of a particular chemotherapeutic agent.

The course of administration of the therapeutic or prophylactic composition may be a single dose or multiple doses. Concurrent administration does not require that the administration of the therapeutic or prophylactic amount of the composition be complete prior to completion of the dose or course of chemotherapy or ionizing radiation. The definition only pertains to initiation of the administration of the therapeutic or prophylactic amount of the composition in relation to course of ionizing radiation or chemotherapeutic agent.

Various combinations of the composition and chemotherapeutic agent may be employed, for example, if the composition is “A” and the secondary agent, such as radio- or chemotherapy, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the compositions of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described therapies.

c. Consecutive Administration

Consecutive administration is defined herein to include beginning administration of the therapeutic or prophylactic amount of the composition of the present invention either before initiation of the therapy with the ionizing radiation or a chemotherapeutic agent, or following completion of a course of therapy with ionizing radiation or a chemotherapeutic agent. As noted above, a course of therapy with chemotherapy or ionizing radiation may involve multiple doses or administrations over a course of time. Consecutive administration requires initiation of administration of the composition of the present invention either prior to beginning a course of administration of ionizing radiation or chemotherapy, or following completion of a course of ionizing radiation or a course of chemotherapy. Consecutive administration, as defined herein, requires that there be no overlap in the course of administration of the composition and the ionizing radiation or chemotherapeutic agent.

For example, in certain embodiments, consecutive administration involves administration of the prophylactically or therapeutically effective amount of the composition of the present invention to be complete within about 5 minutes, about 10 minutes, about 15 minutes, about 20 minutes, about 30 minutes, about 1 hour, about 6 hours, about 1 day, about 5 days, about 10 days, or about 30 days prior to initiation of a course of ionizing radiation or chemotherapy. Similarly, in other embodiments, consecutive administration involves beginning administration of the prophylactically or therapeutically effective amount of the composition of the present invention within about 5 minutes, about 10 minutes, about 15 minutes, about 20 minutes, about 30 minutes, about 1 hour, about 6 hours, about 1 day, about 5 days, about 10 days, or about 30 days after completion of a course of ionizing radiation or chemotherapy. These time intervals are only by way of example, and are not exhaustive. One of ordinary skill in the art would be familiar with the range of possible time intervals for either consecutive or concurrent administration of the composition with ionizing radiation or chemotherapy.

3. Pharmaceutical Compositions

The phrase “pharmaceutically acceptable” and “pharmaceutical” refer to molecular entities and compositions that do not produce an adverse, allergic or other untoward reaction when administered to an animal, or a human, as appropriate. As used herein, “pharmaceutical composition” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients also can be incorporated into the composition. In addition, the composition can include supplementary inactive ingredients. For instance, the composition for use as a mouthwash may include a flavorant or the composition may contain supplementary ingredients to make the formulation timed-release.

Aqueous compositions of the present invention comprise an effective amount of the compound, dissolved or dispersed in a pharmaceutically acceptable carrier or aqueous medium. Examples of aqueous compositions include a spray or aerosol, a solution for intravenous injection, or ophthalmic solution.

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

Administration of therapeutic compositions according to the present invention will be via any common route so long as the target tissue is available via that route. For example, this includes oral, nasal, buccal, anal, rectal, vaginal, or topical ophthalmic. Such compositions would normally be administered as pharmaceutically acceptable compositions that include physiologically acceptable carriers, buffers or other excipients.

The therapeutic and preventive compositions of the present invention are advantageously administered in the form of liquid solutions or suspensions; solid forms suitable for solution in, or suspension in, liquid prior to topical use may also be prepared. A typical composition for such purpose comprises a pharmaceutically acceptable carrier. For instance, the composition may contain 10 mg, 25 mg, 50 mg or up to about 100 mg of human serum albumin per ml of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Preservatives include antimicrobial agents, anti-oxidants, chelating agents and inert gases. The pH and exact concentration of the various components of the pharmaceutical composition are adjusted according to well-known parameters.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions such as mouthwashes and mouthrinses, suspensions, tablets, pills, capsules, sustained release formulations and/or powders. In certain defined embodiments, oral pharmaceutical compositions will comprise an inert diluent and/or assimilable edible carrier, and/or they may be enclosed in hard and/or soft shell gelatin capsule, and/or they may be compressed into tablets, and/or they may be incorporated directly with the food of the diet. For oral therapeutic administration, the active compounds may be incorporated with excipients and/or used in the form of ingestible tablets, buccal tables, troches, capsules, elixirs, suspensions, syrups, wafers, and/or the like. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

The tablets, troches, pills, capsules and/or the like may also contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. A syrup of elixir may contain the active compounds sucrose as a sweetening agent methyl and/or propyl parabens as preservatives, a dye and/or flavoring, such as cherry and/or orange flavor.

For oral administration the compounds of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobeli's Solution). Alternatively, the active ingredient may be incorporated into an antiseptic wash containing sodium borate, glycerin and potassium bicarbonate. The active ingredient also may be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

One may also use solutions and/or sprays, hyposprays, aerosols and/or inhalants in the present invention for administration. Additional formulations which are suitable for other modes of administration include vaginal suppositories and/or pessaries.

Formulations for other types of administration that is topical include, for example, a cream, suppository, ointment or salve.

4. Dosage

An effective amount of the therapeutic or preventive agent is determined based on the intended goal, for example (i) inhibition of growth of a tumor or (ii) suppression of an inflammatory response at the site of disease in a subject.

The quantity to be administered, both according to number of treatments and dose, depends on the subject to be treated, the state of the subject and the protection desired. Precise amounts of the therapeutic composition also depend on the judgment of the practitioner and are peculiar to each individual. For example, the frequency of application of the composition can be once a day, twice a day, once a week, twice a week, or once a month. Duration of treatment may range from one month to one year or longer. Again, the precise preventive regimen will be highly dependent on the subject, the nature of the risk factor, and the judgment of the practitioner.

In certain embodiments, it may be desirable to provide a continuous supply of the therapeutic compositions to the patient. For topical administrations, repeated application would be employed. For various approaches, delayed release formulations could be used that provide limited but constant amounts of the therapeutic agent over an extended period of time. For internal application, continuous perfusion of the region of interest may be preferred. This could be accomplished by catheterization, post-operatively in some cases, followed by continuous administration of the therapeutic agent. The time period for perfusion would be selected by the clinician for the particular patient and situation, but times could range from about 1-2 hours, to 2-6 hours, to about 6-10 hours, to about 10-24 hours, to about 1-2 days, to about 1-2 weeks or longer. Generally, the dose of the therapeutic composition via continuous perfusion will be equivalent to that given by single or multiple injections, adjusted for the period of time over which the doses are administered.

5. Local and Regional Treatment

One of the prime sources of recurrent cancer is the residual, microscopic disease that remains at the primary tumor site, as well as locally and regionally, following tumor excision. In addition, there are analogous situations where natural body cavities are seeded by microscopic tumor cells. The effective treatment of such microscopic disease would present a significant advance in therapeutic regimens.

Thus, in certain embodiments, a cancer may be removed by surgical excision, creating a “cavity.” Both at the time of surgery and thereafter (periodically or continuously), the therapeutic composition of the present invention is administered to the body cavity. This is, in essence, a “topical” treatment of the surface of the cavity. The volume of the composition should be sufficient to ensure that the entire surface of the cavity is contacted by the expression cassette.

### C. Chemotherapeutic Agents

Certain embodiments of the present invention pertain to methods of protecting normal tissue in a subject from the toxicity associated with treatment of a disease with ionizing radiation or a chemotherapeutic agent. Other embodiments of the present invention pertain to methods of treating a disease or disorder in a subject that involve concurrently or consecutively administering a therapeutically effective amount of a composition that includes one of the compounds of the present invention with ionizing radiation or a chemotherapeutic agent.

As used herein, “chemotherapeutic agent” is broadly defined to include a drug, toxin, compound, composition or biological entity which is used as treatment of a disease. For example, a chemotherapeutic agent can include a drug which is used in the treatment of cancer. A chemotherapeutic agent can also include a drug which is used in the treatment of another disease, including, for example, an autoimmune disease, an inflammatory disease, a neurodegenerative disease, a disease associated with ischemia and reperfusion injury, traumatic injury, atherosclerosis, aging, viral infection, and UV or ionizing radiation-induced injury and tissue damage.

Examples of chemotherapeutic agents include, but are not limited to, cisplatin (CDDP), carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxol, transplatinum, 5-fluorouracil, vincristine, vinblastine and methotrexate or any analog or derivative variant thereof.

Chemotherapeutic agents can have any mechanism of action in the treatment of a disease. For example, some chemotherapeutic, directly cross-link DNA, intercalate into DNA, or lead to chromosomal and mitotic aberrations by affecting nucleic acid synthesis. Examples of agents that damage DNA include compounds that interfere with DNA replication, mitosis, and chromosomal segregation. Examples of these compounds include adriamycin (also known as doxorubicin), VP-16 (also known as etoposide), verapamil, podophyllotoxin, and the like. Widely used in clinical setting for the treatment of neoplasms, these compounds are administered through bolus injections intravenously at doses ranging from 25-75 mg/m² at 21 day intervals for adriamycin, to 35-100 mg/m² for etoposide intravenously or orally.

A further discussion of certain classes of chemotherapeutic agents used in the treatment of cancer is as follows.

1. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents, some of which are discussed below.

a. Busulfan

Busulfan (also known as myleran) is a bifunctional alkylating agent. Busulfan is known chemically as 1,4-butanediol dimethanesulfonate.

Busulfan is not a structural analog of the nitrogen mustards. Busulfan is available in tablet form for oral administration. Each scored tablet contains 2 mg busulfan and the inactive ingredients magnesium stearate and sodium chloride.

Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. It has been shown to be superior to splenic irradiation with respect to survival times and maintenance of hemoglobin levels, and to be equivalent to irradiation at controlling splenomegaly.

b. Chlorambucil

Chlorambucil (also known as leukeran) is a bifunctional alkylating agent of the nitrogen mustard type that has been found active against selected human neoplastic diseases. Chlorambucil is known chemically as 4-[bis(2-chlorethyl)amino] benzenebutanoic acid.

Chlorambucil is available in tablet form for oral administration. It is rapidly and completely absorbed from the gastrointestinal tract. After single oral doses of 0.6-1.2 mg/kg, peak plasma chlorambucil levels are reached within one hour and the terminal half-life of the parent drug is estimated at 1.5 hours. 0.1 to 0.2 mg/kg/day or 3 to 6 mg/m²/day or alternatively 0.4 mg/kg may be used for antineoplastic treatment. Treatment regimes are well know to those of skill in the art and can be found in the “Physicians Desk Reference” and in “Remington's Pharmaceutical Sciences” referenced herein.

Chlorambucil is indicated in the treatment of chronic lymphatic (lymphocytic) leukemia, malignant lymphomas including lymphosarcoma, giant follicular lymphoma and Hodgkin's disease. It is not curative in any of these disorders but may produce clinically useful palliation. Thus, it can be used in combination with troglitazone in the treatment of cancer.

C. Cisplatin

Cisplatin has been widely used to treat cancers such as metastatic testicular or ovarian carcinoma, advanced bladder cancer, head or neck cancer, cervical cancer, lung cancer or other tumors. Cisplatin can be used alone or in combination with other agents, with efficacious doses used in clinical applications of 15-20 mg/m² for 5 days every three weeks for a total of three courses. Exemplary doses may be 0.50 mg/m², 1.0 mg/m², 1.50 mg/m², 1.75 mg/m², 2.0 mg/m², 3.0 mg/m², 4.0 mg/m², 5.0 mg/m², 10 mg/m². Of course, all of these dosages are exemplary, and any dosage in-between these points is also expected to be of use in the invention.

Cisplatin is not absorbed orally and must therefore be delivered via injection intravenously, subcutaneously, intratumorally or intraperitoneally.

d. Cyclophosphamide

Cyclophosphamide is 2H-1,3,2-Oxazaphosphorin-2-amine, N,N-bis(2-chloroethyl)tetrahydro-, 2-oxide, monohydrate; termed Cytoxan available from Mead Johnson; and Neosar available from Adria. Cyclophosphamide is prepared by condensing 3-amino-1-propanol with N,N-bis(2-chlorethyl) phosphoramidic dichloride [(ClCH₂CH₂)₂N—POCl₂] in dioxane solution under the catalytic influence of triethylamine. The condensation is double, involving both the hydroxyl and the amino groups, thus effecting the cyclization.

Unlike other β-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable doses for adults include, orally, 1 to 5 mg/kg/day (usually in combination), depending upon gastrointestinal tolerance; or 1 to 2 mg/kg/day; intravenously, initially 40 to 50 mg/kg in divided doses over a period of 2 to 5 days or 10 to 15 mg/kg every 7 to 10 days or 3 to 5 mg/kg twice a week or 1.5 to 3 mg/kg/day. A dose 250 mg/kg/day may be administered as an antineoplastic. Because of gastrointestinal adverse effects, the intravenous route is preferred for loading. During maintenance, a leukocyte count of 3000 to 4000/mm³ usually is desired. The drug also sometimes is administered intramuscularly, by infiltration or into body cavities. It is available in dosage forms for injection of 100, 200 and 500 mg, and tablets of 25 and 50 mg the skilled artisan is referred to “Remington's Pharmaceutical Sciences” 15th Edition, chapter 61, incorporate herein as a reference, for details on doses for administration.

e. Melphalan

Melphalan, also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine.

Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL-) form is known as merphalan or sarcolysin. Melphalan is insoluble in water and has a pKa₁ of ˜2.1. Melphalan is available in tablet form for oral administration and has been used to treat multiple myeloma.

Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of 0.2 mg/kg daily for five days as a single course. Courses are repeated every four to five weeks depending upon hematologic tolerance (Smith and Rutledge, 1975; Young et al., 1978). Alternatively the dose of melphalan used could be as low as 0.05 mg/kg/day or as high as 3 mg/kg/day or any dose in between these doses or above these doses. Some variation in dosage will necessarily occur depending on the condition of the subject being treated. The person responsible for administration will, in any event, determine the appropriate dose for the individual subject.

2. Antimetabolites

Antimetabolites disrupt DNA and RNA synthesis. Unlike alkylating agents, they specifically influence the cell cycle during S phase. They have used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

a. 5-Fluorouracil

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the syntheses of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

3. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms these compounds are administered through bolus injections intravenously at doses ranging from 25-75 mg/m² at 21 day intervals for adriamycin, to 35-100 mg/m² for etoposide intravenously or orally.

a. Doxorubicin

Doxorubicin hydrochloride, 5,12-Naphthacenedione, (8s-cis)-10-[(3-amino-2,3,6-trideoxy-α-L-lyxo-hexopyranosyl)oxy]-7,8,9,10-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of ovarian, endometrial and breast tumors, bronchogenic oat-cell carcinoma, non-small cell lung carcinoma, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma soft tissue sarcoma, Ewing's sarcoma, rhabdomyosarcoma and acute lymphocytic leukemia. It is an alternative drug for the treatment of islet cell, cervical, testicular and adrenocortical cancers. It is also an immunosuppressant.

Doxorubicin is absorbed poorly and must be administered intravenously. The pharmacokinetics are multicompartmental. Distribution phases have half-lives of 12 minutes and 3.3 hr. The elimination half-life is about 30 hr. Forty to 50% is secreted into the bile. Most of the remainder is metabolized in the liver, partly to an active metabolite (doxorubicinol), but a few percent is excreted into the urine. In the presence of liver impairment, the dose should be reduced.

Appropriate doses are, intravenous, adult, 60 to 75 mg/m² at 21-day intervals or 25 to 30 mg/m² on each of 2 or 3 successive days repeated at 3- or 4-wk intervals or 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by 50% if the serum bilirubin lies between 1.2 and 3 mg/dL and by 75% if above 3 mg/dL. The lifetime total dose should not exceed 550 mg/m² in patients with normal heart function and 400 mg/m² in persons having received mediastinal irradiation. Alternatively, 30 mg/m² on each of 3 consecutive days, repeated every 4 wk. Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

In the present invention the inventors have employed troglitazone as an exemplary chemotherapeutic agent to synergistically enhance the antineoplastic effects of the doxorubicin in the treatment of cancers. Those of skill in the art will be able to use the invention as exemplified potentiate the effects of doxorubicin in a range of different pre-cancer and cancers.

b. Daunorubicin

Daunorubicin hydrochloride, 5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11 trihydroxy-10-methoxy-, hydrochloride; also termed cerubidine and available from Wyeth. Daunorubicin intercalates into DNA, blocks DAN-directed RNA polymerase and inhibits DNA synthesis. It can prevent cell division in doses that do not interfere with nucleic acid synthesis.

In combination with other drugs it is included in the first-choice chemotherapy of acute myelocytic leukemia in adults (for induction of remission), acute lymphocytic leukemia and the acute phase of chronic myelocytic leukemia. Oral absorption is poor, and it must be given intravenously. The half-life of distribution is 45 minutes and of elimination, about 19 hr. The half-life of its active metabolite, daunorubicinol, is about 27 hr. Daunorubicin is metabolized mostly in the liver and also secreted into the bile (ca 40%). Dosage must be reduced in liver or renal insufficiencies.

Suitable doses are (base equivalent), intravenous adult, younger than 60 yr. 45 mg/m²/day (30 mg/m² for patients older than 60 yr.) for 1, 2 or 3 days every 3 or 4 wk or 0.8 mg/kg/day for 3 to 6 days every 3 or 4 wk; no more than 550 mg/m² should be given in a lifetime, except only 450 mg/m² if there has been chest irradiation; children, 25 mg/m² once a week unless the age is less than 2 yr. or the body surface less than 0.5 m, in which case the weight-based adult schedule is used. It is available in injectable dosage forms (base equivalent) 20 mg (as the base equivalent to 21.4 mg of the hydrochloride). Exemplary doses may be 10 mg/n², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

c. Mitomycin

Mitomycin (also known as mutamycin and/or mitomycin-C) is an antibiotic isolated from the broth of Streptomyces caespitosus which has been shown to have antitumor activity. The compound is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed.

In humans, mitomycin is rapidly cleared from the serum after intravenous administration. Time required to reduce the serum concentration by 50% after a 30 mg. bolus injection is 17 minutes. After injection of 30 mg., 20 mg., or 10 mg. I.V., the maximal serum concentrations were 2.4 mg./mL, 1.7 mg./mL, and 0.52 mg./mL, respectively. Clearance is effected primarily by metabolism in the liver, but metabolism occurs in other tissues as well. The rate of clearance is inversely proportional to the maximal serum concentration because, it is thought, of saturation of the degradative pathways. Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar.

d. Actinomycin D

Actinomycin D (Dactinomycin) [50-76-0]; C₆₂H₈₆N₁₂O₁₆ (1255.43) is an antineoplastic drug that inhibits DNA-dependent RNA polymerase. It is a component of first-choice combinations for treatment of choriocarcinoma, embryonal rhabdomyosarcoma, testicular tumor and Wilms' tumor. Tumors that fail to respond to systemic treatment sometimes respond to local perfusion. Dactinomycin potentiates radiotherapy. It is a secondary (efferent) immunosuppressive.

Actinomycin D is used in combination with primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hr. The drug does not pass the blood-brain barrier. Actinomycin D is supplied as a lyophilized powder (0.5 mg in each vial). The usual daily dose is 10 to 15 mg/kg; this is given intravenously for 5 days; if no manifestations of toxicity are encountered, additional courses may be given at intervals of 3 to 4 weeks. Daily injections of 100 to 400 mg have been given to children for 10 to 14 days; in other regimens, 3 to 6 mg/kg, for a total of 125 mg/kg, and weekly maintenance doses of 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

e. Bleomycin

Bleomycin is a mixture of cytotoxic glycopeptide antibiotics isolated from a strain of Streptomyces verticillus. Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of >35 mL per minute, the serum or plasma terminal elimination half-life of bleomycin is approximately 115 minutes. In patients with a creatinine clearance of <35 mL per minute, the plasma or serum terminal elimination half-life increases exponentially as the creatinine clearance decreases. In humans, 60% to 70% of an administered dose is recovered in the urine as active bleomycin. Bleomycin may be given by the intramuscular, intravenous, or subcutaneous routes. It is freely soluble in water.

Bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), skin, penis, cervix, and vulva. It has also been used in the treatment of lymphomas and testicular carcinoma.

Because of the possibility of an anaphylactoid reaction, lymphoma patients should be treated with two units or less for the first two doses. If no acute reaction occurs, then the regular dosage schedule may be followed.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

4. Corticosteroid Hormones

Corticosteroid hormones are useful in treating some types of cancer (lymphoma, leukemias, and multiple myeloma). Though these hormones have been used in the treatment of many non-cancer conditions, they are considered chemotherapy drugs when they are implemented to kill or slow the growth of cancer cells. Like troglitazone, corticosteroid hormones can increase the effectiveness of other chemotherapy agents, and consequently, they are frequently used in combination treatments. Prednisone and dexamethasone are examples of corticosteroid hormones.

5. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, vinblastine, vincristine, and vinorelbine.

a. Etoposide (VP16)

VP16 is also known as etoposide and is used primarily for treatment of testicular tumors, in combination with bleomycin and cisplatin, and in combination with cisplatin for small-cell carcinoma of the lung. It is also active against non-Hodgkin's lymphomas, acute nonlymphocytic leukemia, carcinoma of the breast, and Kaposi's sarcoma associated with acquired immunodeficiency syndrome (AIDS).

VP16 is available as a solution (20 mg/ml) for intravenous administration and as 50-mg, liquid-filled capsules for oral use. For small-cell carcinoma of the lung, the intravenous dose (in combination therapy) is can be as much as 100 mg/m² or as little as 2 mg/m², routinely 35 mg/m², daily for 4 days, to 50 mg/m², daily for 5 days have also been used. When given orally, the dose should be doubled. Hence the doses for small cell lung carcinoma may be as high as 200-250 mg/m². The intravenous dose for testicular cancer (in combination therapy) is 50 to 100 mg/m² daily for 5 days, or 100 mg/m² on alternate days, for three doses. Cycles of therapy are usually repeated every 3 to 4 weeks. The drug should be administered slowly during a 30- to 60-minute infusion in order to avoid hypotension and bronchospasm, which are probably due to the solvents used in the formulation.

b. Taxol

Taxol is an antimitotic agent, isolated from the bark of the ash tree, Taxus brevifolia. It binds to tubulin (at a site distinct from that used by the vinca alkaloids) and promotes the assembly of microtubules. Taxol is currently being evaluated clinically; it has activity against malignant melanoma and carcinoma of the ovary. Maximal doses are 30 mg/m² per day for 5 days or 210 to 250 mg/m² given once every 3 weeks. Of course, all of these dosages are exemplary, and any dosage in-between these points is also expected to be of use in the invention.

c. Vinblastine

Vinblastine is another example of a plant alkyloid that can be used in combination with troglitazone for the treatment of cancer and precancer. When cells are incubated with vinblastine, dissolution of the microtubules occurs.

Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, drug disappears from plasma with half-lives of approximately 1 and 20 hours. Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. The drug is given intravenously; special precautions must be taken against subcutaneous extravasation, since this may cause painful irritation and ulceration. The drug should not be injected into an extremity with impaired circulation. After a single dose of 0.3 mg/kg of body weight, myelosuppression reaches its maximum in 7 to 10 days. If a moderate level of leukopenia (approximately 3000 cells/mm³) is not attained, the weekly dose may be increased gradually by increments of 0.05 mg/kg of body weight. In regimens designed to cure testicular cancer, vinblastine is used in doses of 0.3 mg/kg every 3 weeks irrespective of blood cell counts or toxicity.

The most important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, neuroblastoma, and Letterer-Siwe disease (histiocytosis X), as well as in carcinoma of the breast and choriocarcinoma in women.

Doses of vinblastine will be determined by the clinician according to the individual patients need. 0.1 to 0.3 mg/kg can be administered or 1.5 to 2 mg/m² can also be administered. Alternatively, 0.1 mg/m², 0.12 mg/m², 0.14 mg/m², 0.15 mg/m², 0.2 mg/m², 0.25 mg/m², 0.5 mg/m², 1.0 mg/m², 1.2 mg/m², 1.4 mg/m², 1.5 mg/m², 2.0 mg/m², 2.5 mg/m², 5.0 mg/m², 6 mg/m², 8 mg/m², 9 mg/m², 10 mg/m², 20 mg/m², can be given. Of course, all of these dosages are exemplary, and any dosage in-between these points is also expected to be of use in the invention.

d. Vincristine

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents.

Unpredictable absorption has been reported after oral administration of vinblastine or vincristine. At the usual clinical doses the peak concentration of each drug in plasma is approximately 0.4 mM.

Vinblastine and vincristine bind to plasma proteins. They are extensively concentrated in platelets and to a lesser extent in leukocytes and erythrocytes.

Vincristine has a multiphasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilimbin in plasma is greater than 3 mg/dl (about 50 mM).

Vincristine sulfate is available as a solution (1 mg/ml) for intravenous injection. Vincristine used together with corticosteroids is presently the treatment of choice to induce remissions in childhood leukemia; the optimal dosages for these drugs appear to be vincristine, intravenously, 2 mg/m² of body-surface area, weekly, and prednisone, orally, 40 mg/m², daily. Adult patients with Hodgkin's disease or non-Hodgkin's lymphomas usually receive vincristine as a part of a complex protocol. When used in the MOPP regimen, the recommended dose of vincristine is 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisone, and procarbazine (the so-called MOPP regimen), it is the preferred treatment for the advanced stages (I and IV) of this disease. In non-Hodgkin's lymphomas, vincristine is an important agent, particularly when used with cyclophosphamide, bleomycin, doxorubicin, and prednisone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, and carcinomas of the breast, bladder, and the male and female reproductive systems.

Doses of vincristine for use will be determined by the clinician according to the individual patients need. 0.01 to 0.03 mg/kg or 0.4 to 1.4 mg/m² can be administered or 1.5 to 2 mg/m² can also be administered. Alternatively 0.02 mg/m², 0.05 mg/m², 0.06 mg/m², 0.07 mg/m², 0.08 mg/m², 0.1 mg/m², 0.12 mg/m², 0.14 mg/m², 0.15 mg/m², 0.2 mg/m², 0.25 mg/m² can be given as a constant intravenous infusion. Of course, all of these dosages are exemplary, and any dosage in-between these points is also expected to be of use in the invention.

6. Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.

a. Carmustine

Carmustine (sterile carmustine) is one of the nitrosoureas used in the treatment of certain neoplastic diseases. It is 1,3bis(2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended. The structural formula is:

Sterile carmustine is commonly available in 100 mg single dose vials of lyophilized material.

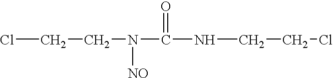

Although

it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisone to treat multiple myeloma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

The recommended dose of carmustine as a single agent in previously untreated patients is 150 to 200 mg/m² intravenously every 6 weeks. This may be given as a single dose or divided into daily injections such as 75 to 100 mg/m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention for example 10 mg/m², 20 mg/m², 30 mg/m² 40 mg/m² 50 mg/m² 60 mg/m² 70 mg/m² 80 mg/m² 90 mg/m² 100 mg/m². The skilled artisan is directed to, “Remington's Pharmaceutical Sciences” 15th Edition, chapter 61. Some variation in dosage will necessarily occur depending on the condition of the subject being treated. The person responsible for administration will, in any event, determine the appropriate dose for the individual subject.

b. Lomustine

Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (0.05 mg per mL) and in absolute alcohol (70 mg per mL). Lomustine is relatively insoluble in water (<0.05 mg per mL). It is relatively unionized at a physiological pH. Inactive ingredients in lomustine capsules are: magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from 30 mg/m² to 100 mg/m², about half of the radioactivity given was excreted in the form of degradation products within 24 hours. The serum half-life of the metabolites ranges from 16 hours to 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is 130 mg/m² as a single oral dose every 6 weeks. In individuals with compromised bone marrow function, the dose should be reduced to 100 mg/m² every 6 weeks. When lomustine is used in combination with other myelosuppressive drugs, the doses should be adjusted accordingly. It is understood that other doses may be used for example, 20 mg/m² 30 mg/m², 40 mg/m², 50 mg/m², 60 mg/m², 70 mg/m², 80 mg/m², 90 mg/m², 100 mg/m², 120 mg/m² or any doses between these figures as determined by the clinician to be necessary for the individual being treated.

7. Miscellaneous Agents

Some chemotherapy agents do not qualify into the previous categories based on their activities. However, it is contemplated that they are included within the method of the present invention for use in combination therapies of cancer with troglitazone. They include amsacrine, L-asparaginase, tretinoin, and Tumor Necrosis Factor (TNF), some of which are discussed below.

a. Tumor Necrosis Factor

Tumor Necrosis Factor [TNF; Cachectin] is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TINF and interferon-a also has been found to possess anti-cancer activity.

In addition to combination treatment therapies comprising troglitazone or thiazolidinediones and another chemotherapeutic agent, it is also contemplated that the present invention includes the use of sex hormones according to the methods described herein in the treatment of cancer. While this method is not limited to the treatment of a specific cancer, this use of hormones in this combination therapy has benefits with respect to cancers of the breast, prostate, and endometrial (lining of the uterus). Examples of these hormones are estrogens, anti-estrogens, progesterones, and androgens.

### D. UV and Ionizing Radiation

Certain embodiments of the present invention pertain to methods of protecting normal tissue in a subject from the toxicity associated with treatment of a disease with ionizing radiation. Other embodiments of the present invention pertain to methods of to methods of treating a disease that involve concurrently or consecutively administering a therapeutically effective amount of a compound of the present invention and ionizing radiation.

UV-radiation is defined herein to include radiation that induces DNA damage by UV waves. Ionizing radiation is defined herein to include radiation and waves that induce DNA damage through the use of, for example, γ-irradiation, radioisotopes, and the like. UV-radiation and ionizing radiation are commonly used in the therapy of disease, such as cancer. Therapy may be achieved by irradiating the localized tumor site with the above described forms of radiation. It is most likely that all of these factors effect a broad range of damage DNA, on the precursors of DNA, the replication and repair of DNA, and the assembly and maintenance of chromosomes. As used herein, treatment with UV-radiation and ionizing radiation is not limited to cancer, but can include treatment of other diseases. One of ordinary skill in the art would be familiar with the clinical indications for use of these forms of radiation.

One of ordinary skill in the art would be familiar with the dosage range of UV or ionizing radiation that are required in the treatment of a particular disease process, such as cancer. Dosage ranges for ionizing radiation that uses radioisotopes may vary widely, and depend on the half-life of the isotope, the strength and type of radiation emitted, and the uptake by the neoplastic cells.

### E. Other Secondary Therapies

Certain embodiments of the present invention pertain to methods of treating a disease or disorder in a subject that include concurrently or consecutively administering a therapeutically effective amount of the composition and ionizing radiation or a chemotherapeutic agent to the subject. Ionizing radiation and chemotherapeutic agents have been previously discussed. Concurrent and consecutive administration have also been previously discussed.

The subject, as noted above, can be afflicted with any disease or disorder. One of ordinary skill in the art would be familiar with the wide range of diseases and disorders that are amenable to treatment with a chemotherapeutic agent or ionizing radiation. For example, the subject may be a cancer patient.

In certain embodiments of the present methods, the subject who is to receive the therapeutic composition of the present invention may not only receive treatment with ionizing radiation or a chemotherapeutic agent, but may also be receiving treatment with another modality. For example, if the patient is a cancer patient, the patient may be receiving treatment with surgery or gene therapy.

Surgical treatment for removal of an abnormal growth, such as a cancer, is a common therapeutic method. This attempts to remove the entire abnormal growth. In the case of cancer, surgery is generally combined with chemotherapy and/or radiotherapy to ensure the destruction of any remaining neoplastic or malignant cells. Thus, in the context of the present invention surgery may be used in addition to using the therapeutic compositions, chemotherapy, and ionizing radiation of the present invention.

In the case of surgical intervention, the compositions of the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising a the therapeutic composition. The perfusion may be continued post-resection, for example, by leaving a catheter implanted at the site of the surgery. Periodic post-surgical treatment also is envisioned.

In certain embodiments, the tumor being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently may be described in terms of plaque forming units (pfi) for a viral construct. Unit doses range from 10³, 10⁴, 10⁵, 10⁶, 10⁷, 10⁸, 10⁹, 10¹⁰, 10¹¹, 10¹², 10¹³ pfu and higher.

Immunotherapeutics, generally, rely on the use of immune effector cells and molecules to target and destroy cancer cells. Methods of the present invention may involve treatment of subjects who are concurrently receiving immunotherapy for a disease.

Immunotherapy, thus, could be used as part of a combined therapy, in conjunction with therapy using the compositions of the present invention.

Secondary treatments may also include gene therapy. One of ordinary skill in the art would be familiar with treatment options involving gene therapy.

Examples of other types of therapies include, cryotherapy, toxin therapy, or hormonal therapy. One of skill in the art would know that this list is not exhaustive of the types of treatment modalities available for diseases, such as cancer.

### F. Protection of Normal Tissue from Toxicity of Ionizing Radiation and Chemotherapy

As noted above, certain embodiments of the present invention pertain to methods of protecting normal tissue in a subject from the toxicity association with treatment of a disease with ionizing radiation or a chemotherapeutic agent, involving concurrently or consecutively administering to the subject a prophylactically effective amount of the composition and the ionizing radiation or chemotherapeutic agent.

Concurrent and consecutive administration have been discussed and defined above. One of ordinary skill in the art would be familiar with methods of administration and dosing for optimizing protection of normal tissue from the toxicity associated with ionizing radiation and chemotherapy. The dose and method of administration will in large part be related to the particular disease process, and to the required course of ionizing radiation or chemotherapy that is necessary to treat the disease. In some embodiments, a single dose of the composition will be sufficient, whereas in other embodiments, a course of the composition involving multiple doses over a prolonged period of time may be required.

For example, in some embodiments, administration of the prophylactic composition may be conducted such that it is complete within about 5 minutes, about 10 minutes, about 30 minutes, about 1 hour, or about 6 hours prior to initiation of a dose of radiation therapy or chemotherapy. In other embodiments, the prophylactically effective amount of the composition is administered concurrently with the ionizing radiation or chemotherapeutic agent. One of ordinary skill in the art would be able to design an appropriate regimen involving the composition such that it would be most effective in preventing the toxicity associated with treatment of a disease with ionizing radiation or a chemotherapeutic agent.

### G. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Chemicals: Anhydrous acetonitrile, paraformaldehyde, zinc chloride, acetyl chloride, chloromethyl butyrate, and chloromethyl pivalate were purchased from Fisher Scientific (Pittsburgh, Pa.). HPLC grade methanol was obtained from Curtin Matheson Scientific Inc. (Houston, Tex.). Porcine liver esterase (PLE, EC 3.1.1.1) was purchased from Sigma (St. Louis, Mo.). Porcine brain L-α-phosphatidylcholine (PC) and NBD C₆-ceramide were obtained from Avanti Polar Lipids (Alabaster, Ala.) and Molecular Probes (Eugene, Oreg.), respectively.

Synthesis and characterization of S-(alkoxyacyl) D609 prodrugs: D609 was synthesized and purified as described by Rao (Rao, 1971), and its purity was determined to be >97%. Chloromethyl acetate was prepared as described by Bodor et al. (Bodor et al., 1983). The prodrugs of D609 were prepared and purified as illustrated in FIG. 2 and described below:

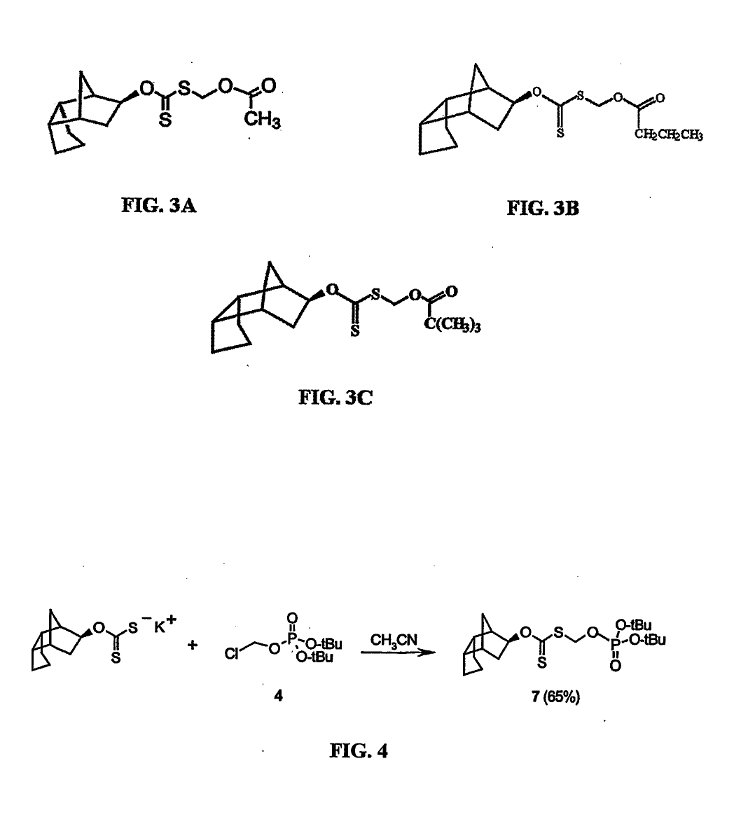

S-methyleneoxyacetyl D609 (prodrug 1) (FIG. 3A): Chloromethyl acetate (41 mg, 0.38 mmol) was added to a solution of D609 (100 mg, 0.38 mmol) in 15 ml anhydrous acetonitrile under nitrogen. The reaction mixture was stirred at room temperature for 8 h and then placed under reduced pressure to remove the solvent. The resulting suspension was extracted with dichloromethane (3×9 ml), the organic solutions combined, and the solvent evaporated. The resulting oil was separated by silica gel column chromatography (ethyl acetate:hexane=1:10) to yield the target compound as a yellow oil (70 mg, 64%). ¹H-NMR and ¹³C-NMR spectra were obtained using a Varian Inova-400 MHz NMR instrument (Palo Alto, Calif.) with tetramethylsilane as internal standard. ¹H-NMR (CDCl₃, 400 MHz, δ ppm): 5.59 (s, 2H, CH₂), 5.44 (d, 1H, CH, J=10.0 Hz), 2.45-1.46 (m, 14H), 2.06 (s, 3H, CH₃). ¹³C-NMR (CDCl₃, 100 MHz, δ ppm): 210.35, 170.70, 85.85, 66.74, 47.04, 44.43, 44.17, 41.08, 40.59, 34.12, 28.59, 27.43, 26.60, 21.14.

Alternatively, prodrug 1 was prepared as follows: Chloromethyl acetate was prepared according to the method of Nudelman et al., 2001: a mixture of acetyl chloride (5.00 g, 64 mmol), paraformaldehyde (1.91 g, 64 mmol) and ZnCl₂ (cat.) were mixed together at room temperature. An exothermic reaction occurred after several minutes, whereupon the temperature reached 75-80° C. After the exotherm was completed, the reaction was heated to 75° C. for 3 hrs. The product was isolated by distillation (90-90.5° C., 760 mmHg) to give the product as a colorless oil (1.96 g, 29%). The chloromethyl acetate (0.04 g, 0.38 mmol) was added to a solution of D609 (100 mg, 0.38 mmol) in anhydrous acetonitrile (15 ml) maintained under nitrogen. The reaction was stirred at room temperature for 16 hrs. The mixture was then placed under reduced pressure to remove the solvent and the residue was extracted three times with 10 ml of dichloromethane. The organic solutions were combined and the solvent evaporated. The residue was separated by column chromatography over silica get (ethyl acetate:hexane=1:15) to give the target compound as a yellow color oil (0.070 g, 63.6% yield). Data collected was as follows: ¹H-NMR (CDCl₃, 400 MHz, δ ppm): 5.59 (S, 2H), 5.47(m, 1H), 2.46-1.46 (m, 14H), 2.06 (s, 3H, CH₃). ¹³C-NMR (CDCl₃, 100 MHz, δ ppm): 21.15, 26.60, 27.44, 28.60, 34.12, 40.60, 41.08, 44.17, 44.43, 47.04, 66.74, 85.85, 170.50, 210.10.

S-methyleneoxybutryl D609 (prodrug 2) (FIG. 3B): Prodrug 2 was prepared similarly to S-methyleneoxyacetyl D609 1, except that the commercially available chloromethyl butyrate was used. The yield was 73%. ¹H-NMR (CDCl₃, 400 MHz, δ ppm): 5.58 (s, 2H, CH₂), 5.44 (d, 1H, CH, J=10.0 Hz), 2.45-1.45 (m, 14H), 2.28 (t, 2H, CH₂, J=7.8), 1.67-1.59(m, 4H, 2 CH₂), 0.92-0.88 (t, 3H, CH₃, J=7.4 Hz). ¹³C-NMR (CDCl₃, 100 MHz, δ ppm): 210.23, 173.11, 85.72, 66.51, 47.03, 44.44, 44.18, 41.08, 40.60, 36.17, 34.12, 28.60, 27.44, 26.60, 18.56, 13.91.

S-methyleneoxypivalyl D609 (prodrug 3) (FIG. 3C): It was prepared in a way similar to that of S-methyleneoxyacetyl D609 1, except that the commercially available chloromethyl pivalate was used. The yield was 86%. Data: ¹H-NMR (CDCl₃, 400 MHz, δ ppm): 5.54(s, 2H, CH₂), 5.44 (d, 1H, CH, J=10.0 Hz), 2.46-1.399 (m, 14H), 1.15(s, 9H, 3 CH₃); ¹³C-NMR (CDCl₃, 100 MHz, δ ppm): 210.30, 178.11, 87.87, 66.61, 47.50, 46.122, 42.75, 39.92, 39.229, 39.09, 32.25, 31.93, 30.14, 28.04, 27.24.

Alternatively, prodrug 3 can be prepared in a similar manner to the synthesis of compound 7 in FIG. 4 (i.e., S-(methylenoxy)-D609, di(t-butoxy)phosphoryl), which is discussed below. Chromatography over silica gel (ethyl acetate:hexane=1:25) gave the desired product as a yellow oil (0.030 g, 52.6% yield). Data: ¹H-NMR (CDCl₃, 400 MHz, δ ppm):5.56 (s, 2H), 5.21 (m, 1H), 2.26-0.85 (m, 14H), 1.17 (s, 9H); ¹³C-NMR (CDCl₃, 100 MHz, δ ppm): 27.24, 28.04, 30.14, 31.93, 32.25, 39.09, 39.30, 39.92, 42.75, 46.12, 47.50, 66.61, 87.87, 179.0, 210.10.

Synthesis and Characterization of S-(alkoxyphosphoryl) D609 Prodrugs:

S-(methylenoxy)-D609, di(t-butoxy)phosphoryl (compound 7 in FIG. 4): Di-tert-butyl chloromethyl phosphate was prepared as described by Krise et al., 1999. Di-tert-butyl chloromethyl phosphate (97 mg, 0.38 mmol) was dissolved in acetonitrile (5 ml) and a solution of D609 (100 mg, 0.38 mmol) in acetonitrile (15 ml) was added, while being maintained under nitrogen. The reaction was stirred at room temperature for 12 hrs. The mixture was then placed under reduced pressure to remove solvent. The residue was then extracted three times with 10 ml of dichloromethane. The organic solutions were combined and the solvent evaporated to give the crude product. The residue was separate by column chromatography over silica gel (ethyl acetate:hexane=1:5) to give the target compound 7 as a yellow oil (0.110 g, 64.7%). Data (compound 7): ¹H-NMR (CDCl₃, 400 MHz, δ ppm) 5.59 (m, 1H), 5.42(d, 2H), 2.45-1.54(m, 14H), 1.40(s, 18H); ¹³C-NMR (CDCl₃, 100 MHz, δ ppm) 26.00, 27.00, 28.80, 30.00, 31.50, 34.00, 40.50, 41.00, 44.00, 44.10, 47.00, 59.80, 74.00, 86.00, 210.00.

High-performance liquid chromatography (HPLC) analysis: A reverse-phase HPLC assay was developed for the quantitative analysis of D609 and S-(alkoxyacyl) D609 prodrugs using a Gilson HPLC system (Middleton, Wis.). The system was equipped with a 306-pump and a GAT LCD 501-detector set at 290 nm for the analysis. A 3.9×150 mm Nova-Pack C18 column (5 μm particle size) was used with a mobile phase consisting of 100% methanol at a flow rate of 1.0 ml/min. The retention times were: D609, 0.95±0.01 min; prodrug 1, 1.77±0.03 min; prodrug 2, 1.97±0.01 min; and prodrug 3 2.08±0.01 min.

Detection of D609 with 5,5′-dithiobis(2-nitrobenzoic acid) (DTNB): DTNB is a commonly used reagent for the detection of free thiol compounds (Lauderback et al., 2003). DTNB stock solution was prepared in PBS and added in excess to a sample containing D609 as specified in figure legends. D609 rapidly reacts with DTNB to produce a mixed disulfide plus the stable thiolate anion, 5-thio-2-nitrobenzoate (TNB), which can be quantified by measuring the OD value at 412 nm using a Vmax plate reader (Molecular Devices, Sunnyvale, Calif.), as described before (Lauderback et al., 2003). The concentration of D609 (paM) was calculated based on a linear D609-DTNB standard curve.

Cell culture: Human monocytic leukemia U937 cells were originally obtained from ATCC (Manassas, Va.) and were cultured in complete medium (RPMI 1640 medium supplemented with 10% FBS, 2 mM L-glutamine, 100 unit/ml penicillin, and 100 μg/ml streptomycin). The cells in exponential growth phase were harvested from cultures and used in all of the experiments. In all cell cultures with D609 prodrug, no exogenous esterase was added as FBS contains about 1 unit/ml esterases that efficiently hydrolyze the prodrug (data not shown).

Cell viability assay: The MTT assay was used to quantify viable U937 cells (Hansen et al., 1989). U937 cells were harvested, washed and resuspended in complete medium at a concentration of 5×10⁵ cells per ml. Aliquots (100 μl) of the cell suspension were added to wells of a 96-well microtiter plate with the addition of 100 μl of complete medium (control) or various concentrations of D609 or a D609 prodrug (diluted in complete medium). After 48 h incubation, the plates were centrifuged to remove the supernatants from the culture, and 50 μl of MTT at a concentration of 5 mg/ml in phosphate-buffered saline (PBS) was added to each well. The plates were incubated for 4 h at 37° C. to allow for the formation of a colored formazan. The formazan was solubilized by lysing the cells with 100 μl of lysis buffer containing 20 (w/v) % dodecylsulfate and 50 (v/v) % N,N-dimethyl formamide, pH 4.7. Absorbance of the formazan was measured at 595 nm using a Vmax plate reader (Molecular Devices, Sunnyvale, Calif.). The viability of the cells was expressed as a percent of control calculated by the formula A_{d}/A_{c}×100, where A_{d} and A_{c}, represent the absorbance of drug-treated and untreated control cells, respectively, and expressed as a percentage of control.

Apoptosis Assays: U937 cells (5×10⁵/ml) were cultured with vehicle (0.5% DMSO) or 177 μM D609 or prodrug 2. After 24 h incubation, the cells were harvested, washed and then fixed in 70% ethanol at 4° C. for 24 h. They were stained with a PI staining solution (PBS containing PI 50 μg/ml; RNase A 100 U/ml; and 0.1 mM EDTA) for 2 h at room temperature before flow cytometric analysis (10,000 events/sample). The percentage of apoptotic cells was determined by quantification of the sub-G_{0/1} population using a FACS Caliber (Becton Dickinson, San Jose, Calif.).

Analysis of sphingomyelin synthase (SMS) activity: U937 cells (2×10⁶/ml) were cultured with vehicle (0.5% DMSO) or 177 μM D609 or prodrug 2. After 0.5, 1 and 2 h incubation, the cells were harvested, washed and then homogenized in ice-cold lysis buffer (250 mM sucrose; 5 mM HEPES, pH 7.4; 1 mM phenylmethylsulfonyl fluoride; and 20 μg/ml each of chymostatin, leupeptin, antipain, and pepstatin) by 15 passages through a 27-gauge×0.5-inch needle. The cell lysates were first centrifuged at 1000×g for 10 min at 4° C. to remove all the unbroken cells and nuclei. The resultant supernatants were quantified for protein concentration using the Bio-Rad protein assay reagent (Bio-Rad, Hercules, Calif.) and assayed for SMS activity as follows: aliquots of the cell lysates containing 50 μg protein were preincubated for 10 min at 30° C. in a total volume of 50 μl of incubation buffer (50 mM Tris-HCl, pH 7.4; 25 mM KCl; and 0.5 mM EDTA). The reaction was started by addition of 2 nmol NBD C₆-ceramide and 12 nmol PC to give a final volume of 50 μl and incubated for 30 min. The reaction was stopped by addition of 200 μl chloroform/methanol (1:1, v/v); the mixture was vortexed and kept on ice. The chloroform/methanol fraction was isolated, and the lipids were resolved by TLC (silica gel) in chloroform:methanol:15 mM CaCl₂ (90:52.5:12) (Luberto and Hannun, 1998; Meng et al., 2004). The formation of NBD-C₆-sphingomyelin was quantified by determination of the fluorescent intensity of NBD-C6-sphingomyelin using a phosphoimager. Values for blanks were subtracted from total values of NBD-C₆-sphingmyelin to yield the amount of NBD-C₆-sphingmyelin produced in each sample.

Ceramide analysis: The levels of various species of ceramide were measured using positive mode electrospray ionization (ESI)/MS/MS analysis at the Lipidomics Core facility in the Department of Biochemistry and Molecular Biology at Medical University of South Carolina as described before (Pettus et al., 2003b; Pettus et al., 2003a). ESI/MS/MS analysis of ceramide was performed on a Thermo Finnigan TSQ 7000 triple quadruple mass spectrometer, operating in a multiple reaction monitoring (MRM) positive ionization mode. U937 cells (4×10⁶/sample) were washed twice with PBS after they were harvested from cultures. The cell pellets were dissolved in methanol, and lipids were extracted as previously reported (Luberto and Hannun, 1998; Meng et al., 2004). An aliquot of the lipid extracts was taken for inorganic phosphate determination and the remainder was evaporated to dryness and reconstituted in 100 μL of methanol. The reconstituted samples were injected on the Surveyor/TSQ 7000 LC/MS system and gradient was eluted from the BDS Hypersil C8, 150×3.2 mm, 3 μm particle size column, with 1.0 mM methanolic ammonium formate/2 mM aqueous ammonium formate mobile phase system. Peaks for the target analytes and internal standards were collected and processed using the Xcalibur software system. Various species of ceramide were quantified using N-palmitoyl-D-erythro-sphingosine, C₁₃ base (C₁₃ ceramide) and N-heptadecanoyl-D-erythro-sphingosine, C₁₈ base (C₁₈ ceramide) as internal calibration standards. Calibration curves were constructed by plotting peak area ratios of synthetic standards corresponding to each target analyte with respect to the appropriate internal standard. The target analyte peak areas from the samples were similarly normalized to their respective internal standard then compared with the calibration curves using a linear regression model. The results are expressed as pmole ceramide/nmole lipid phosphate (Pi).

Statistical Analysis: The data were analyzed by analysis of variance. In the event that analysis of variance justified post hoc comparisons between group means, these were conducted using the Student-Newman-Keuls test for multiple comparisons. For experiments in which only single experimental and control groups were used, group differences were examined by unpaired Student's t test. Differences were considered significant at p<0.05.

### Example 2

### D609 is a Potent Antioxidant

D609 is a xanthate derivative that can reversibly dissociate and protonate in solution to form xanthate anions and xanthic acid, respectively (Rao, 1971). In a cell free condition, D609 can inhibit several hydroxyl radical-induced events, including (1) oxidation of DHR and terephthalic acid; (2) formation of the PBN-free radical spin adducts; and (3) lipid peroxidation of synaptosomal membranes.

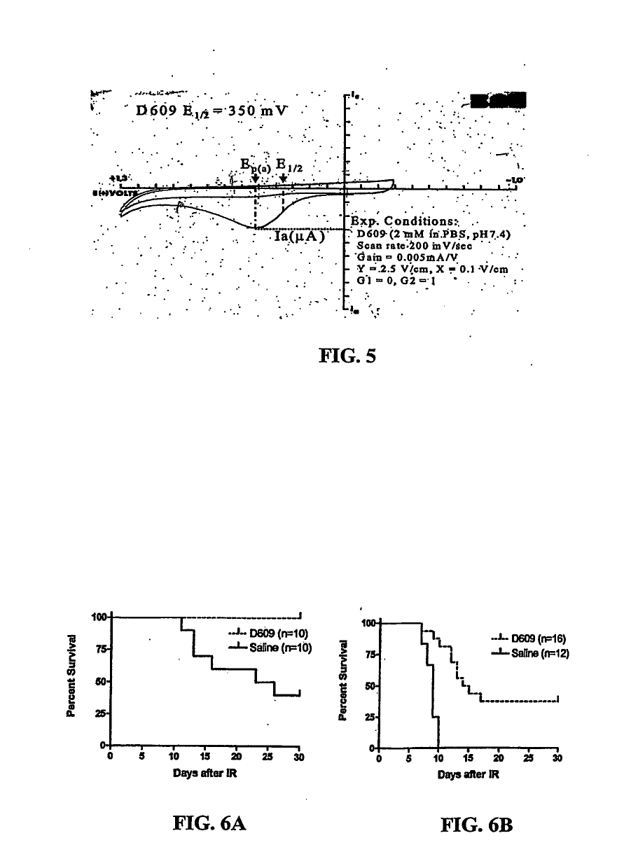

The known reactive oxygen species (ROS) that D609 can scavenge include HO⁻, O₂⁻, and H₂O₂ (Giron-Calle et al., 2002). D609 has the ability to react with other ROS and free radicals, since xanthates generally have a high reduction potential (Rao, 1971). Using cyclic voltammetry, a convenient tool for the evaluation of the antioxidant capacity of various small molecules and biological specimens (Chevion et al., 1997), the reducing power of D609 at physiological pH was measured. D609 was dissolved in PBS (pH 7.4) at 2 mM concentration, and E_{1/2} was measured using a cyclic voltammetry apparatus (Model CV-2, from BAS, West Lafayette, 1N). As shown in FIG. 5, D609 can donate a single electron at the potential of E_{1/2}=350 mV. The E_{1/2} value of D609 is similar to that of vitamin C (E_{1/2}=380 mV) (Chevion et al., 1997). These findings demonstrate that D609 is a novel biological antioxidant.

### Example 3

### D609-Mediated Radiation Protection

It has been shown that various nucleophilic sulfur antioxidants are potent cytoprotectants that can ameliorate IR-induced oxidative stress and tissue damage. Thus, whether D609 also functions as an effective radioprotectant was investigated.

BALB/c mice were exposed to 6.5 or 8.5 Gy total body IR 10 minutes after they received a single dose (50 mg/kg) of iv injection of D609 or vehicle (saline) through the tail vein. The death of these mice was recorded during a 30-day observation period after IR.

It was found that pre-incubation of lymphocytes with D609 resulted in a significant diminution of several IR-induced events, including: 1) production of ROS; 2) decrease in intracellular reduced GSH; 3) oxidative damage to proteins and lipids; and 4) activation of NF-κB. Moreover, when D609 (50 mg/kg, iv) was administered to mice 10 min prior to total body

IR, it protected the mice from IR-induced lethality (FIG. 6A, FIG. 6B). However, incubation of various tumor cells with D609 failed to protect them from IR-induced apoptosis. Instead, D609 exhibited selective cytotoxicity against these cells and enhanced IR-induced tumor cell apoptosis. These results indicate that D609 is not only a potent antioxidant but also functions as an effective cytoprotectant that has the ability to selectively inhibit IR-induced normal cell oxidative damage and protect mice from IR-induced lethality.

### Example 4

### D609 is a Selective Antitumor Agent but a Normal Immune Response Enhancer

Unlike other nucleophilic sulfur antioxidants, D609 is also a potent and selective antitumor agent. Previously, it was shown that D609 kills transformed and malignant cells but has little, if any, toxicity against normal cells in vitro and in vivo. Studies were conducted to determine whether D609 selectively induces tumor cell death by apoptosis.

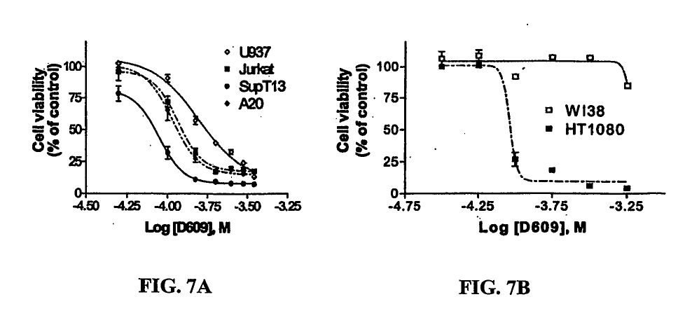

The cell viability was analyzed by MTT assay after various leukemia cells (U937, Jurkat, SupT13 and A20), solid tumor cells (HT1080) and normal human fibroblasts (WI38) were cultured with vehicle or different concentrations of D609 for 48 hours. The results, shown in FIG. 7A and FIG. 7B, are expressed as a percent of vehicle control and presented as means±SEM (n=3).

Both leukemia and solid tumor cells incubated with D609 exhibited a dose-dependent reduction in cell viability, while normal human fibroblasts (WI38) were relatively resistant to D609 treatment (FIG. 7A, FIG. 7B). Using the annexin V-FITC staining and flow cytometric analysis, it was found that D609 primarily induces tumor cell death by apoptosis. This was also confirmed by the analysis of sub-G_{0/1} cells, which measures D609-induced DNA fragmentation. In addition, when D609 was combined with daunorubicin, mitoxantrone, TNFa, or ant-Fas antibody, it enhanced their tumor cell cytotoxicity (Amtmann and Sauer, 1990; Bettaieb et al., 1999; Pron-Ares et al., 1997).

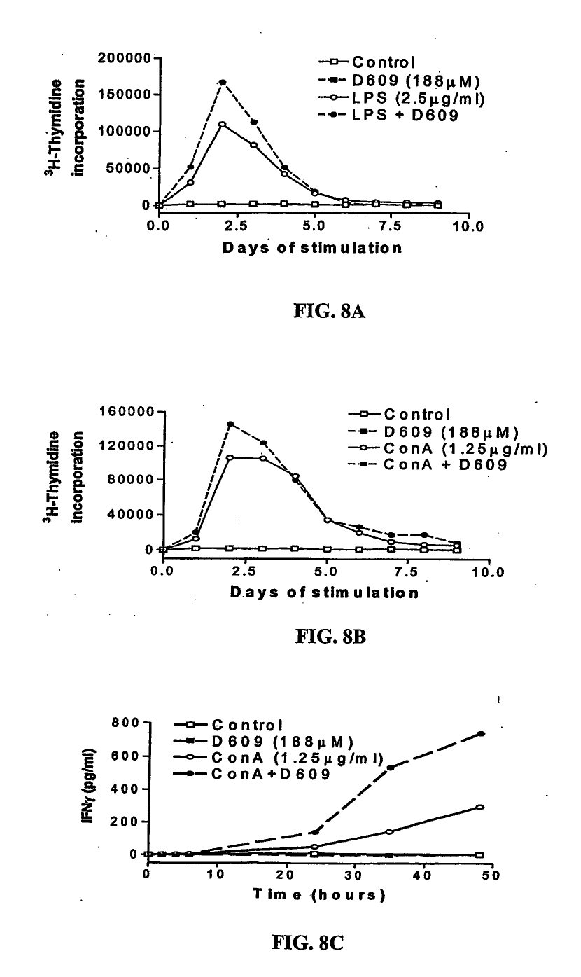

Studies were conducted to determine whether D609 enhances mouse splenic lymphocyte mitogenic responses and IFNγ production. Mouse splenic lymphocytes at 2.5×10⁶/ml were stimulated with LPS or ConA in the presence or absence of D609. The cell proliferation was measure by 3H-thymidine incorporation after the cells were cultured with LPS (FIG. 8A) or with ConA (FIG. 8B). Similar results on D609-induced enhancement of lymphocyte proliferation also were observed in cells stimulated either lower or higher concentrations of LPS or ConA. In addition, the supernatants of ConA-stimulated lymphocyte cultures were harvested at various times and measured for IFNγ production by ELISA (FIG. 8C).

As shown in FIG. 8A-FIG. 8C, D609 actually enhanced normal lymphocyte proliferation in response to mitogen stimulation. As shown in FIG. 8, incubation of normal mouse splenic lymphocytes with D609 (188 μM=50 μg/ml) enhanced their mitogenic responses to stimulation with LPS (a B cell mitogen) or ConA (a T cell mitogen). The enhancement was seen in the cells stimulated with various concentrations of LPS (from 0.75 to 10 μg/ml) or ConA (from 0.3 to 10 μg/ml). The greatest enhancement was seen in the cells that were stimulated with a sub-optimal concentration of LPS (2.5 μg/ml) or ConA (1.25 μg/ml) (FIG. 8A, FIG. 8B). In addition, the production of various cytokines, particularly interferon-γ (IFNγ), was significantly augmented by D609 (FIG. 8C). These results suggest that D609 not only acts as selective tumor cytotoxic agent but also may function as an immune modulator that enhances lymphocyte-mediated immune reactions. Considering that IFNγ is one of the major cytokines regulating T and NK cell-mediated antitumor activities, D609 may have the potential to enhance antitumor immune responses.

### Example 5

### Mechanisms of Antitumor Action of D609

To determine if inhibition of PC-PLC or SMS is responsible for D609 antitumor action, studies were conducted to compare the inhibitory effects of D609, cyclohexyl xanthate and tricyclodecanol on PC-PLC and SMS and the relationship of this inhibition to tumor cytotoxicity.

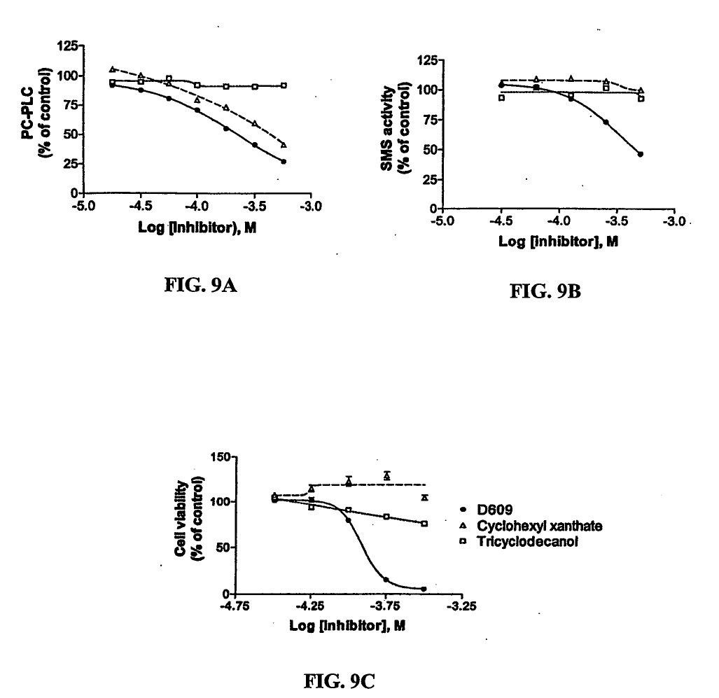

In this study, p-nitrophenyl-phosphorylcholine (pNPP, 40 mM) was incubated with 2 U/ml phospholipase C (Type XI from B. cereus) in the presence or absence of various concentrations of inhibitors for 2 hrs at 37° C. The amount of the cleaved substrate pNPP was quantified by determination of the optical density at 410 nm using an ELISA reader. The results are expressed as percent of vehicle control and presented as means±SEM (n=3) (FIG. 9A). NBD-C₆-ceramide and PC were incubated with U937 cell lysates containing 50 μg protein in the presence or absence of various concentrations of inhibitors for 30 min at 30° C. The formation of NBD-C₆-sphingomyelin as analyzed by TLC and quantified by determination of the fluorescent intensity of NBD-C6-sphingomyelin using a phosphorimager. The results are expressed as percent of vehicle control (FIG. 9B). U937 cells were incubated with different concentrations of inhibitors for 48 hours. The cell viability was analyzed by MTT assay and the results are expressed as percent of vehicle control and presented as means±SEM (n=3) (FIG. 9C).

Both D609 and cyclohexyl xanthate dose-dependently inhibited the activity of the Bacillus cereus bacteria-derived PC-PLC as described previously (FIG. 9A; Amtmann, 1996). Using a cell lysate-based in vitro assay system (Luberto and Hannun, 1998; Riboni et al., 2001), it was found that only D609 inhibited the SMS activity in a dose-dependent manner, while cyclohexyl xanthate had no effect (FIG. 9B). Tricyclodecanol, the alcohol used to synthesize D609, is devoid of any of these activities (FIG. 9A, FIG. 9B). When U937 cells were incubated with different concentrations of these compounds, it was found that only D609 induced significant cell death in U937 cells in a dose-dependent manner whereas the other two compounds had no or only modest effects on the cell viability. These results suggest that D609 may induce tumor cell death primarily by inhibiting SMS activity.

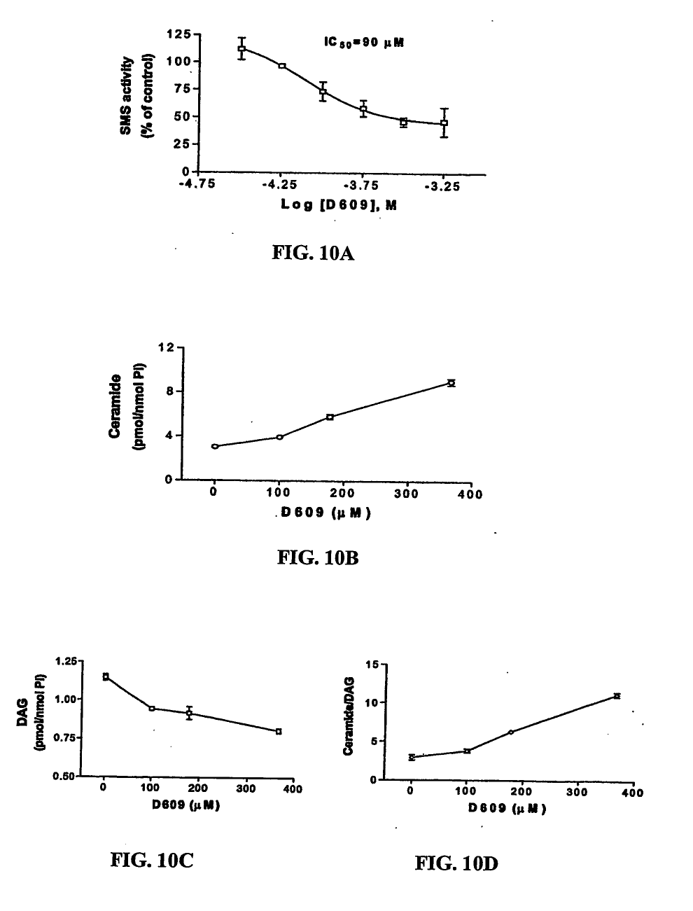

To validate if inhibition of SMS may mediate D609-induced tumor cell death, the cellular level of SMS activity in U937 cells treated with vehicle or D609 were studied. Cell lysates were prepared after U937 cells were cultured with vehicle or different concentrations of D609 for 2 hrs when the cell death was undetectable (FIG. 10A). NBD-C₆-ceramide and PC were incubated with the cell lysates containing 50 μg proteins for 30 min at 30° C. The formation of NBD-C₆-sphingomyelin was analyzed by TLC and quantified by determination of the fluorescent intensity of NBD-C₆-sphingomyelin using a phosphoimager. The results, shown in FIGS. 10B-10D, are expressed as percent of the control cultures with vehicle and presented as means±SEM (n=3). Lipids were extracted from U937 cells after the cells were cultured with vehicle or different concentrations of D609 for 2 hrs. The levels of ceramide (FIG. 10B) and DAG (FIG. 10C) were analyzed by the DAG kinase assay, normalized to the levels of cellular phospholipid and expressed as pmole/nmole phophate (pi) or the ratio of ceramide and DAG (FIG. 10D). The results represent means±SEM (n=3).

As shown in FIG. 10A, U937 cells incubated with different concentrations of D609 exhibited a dose-dependent reduction in SMS activity. The IC₅₀ value is about 90 μM (or 23.8 μg/ml). Since SMS transfers the PhoCho group from PC to ceramide, generating DAG and SM, SMS has the ability of simultaneously regulating the intracellular levels of DAG and ceramide in opposite directions (Luberto and Hannun, 1998). Inhibition of SMS by D609 should increase the intracellular level of ceramide while decreasing that of DAG, which could result in an elevation of the ratio between these two important intracellular signal molecules that regulate cell proliferation or cell cycle arrest and senescence, survival and cell death. Indeed, as shown in FIGS. 10B-10D, U937 cells treated with different concentrations of D609 showed an increase in the level of ceramide and a decrease in that of DAG in a dose-dependent manner. Correspondingly, the ratio between ceramide and DAG was dramatically elevated.

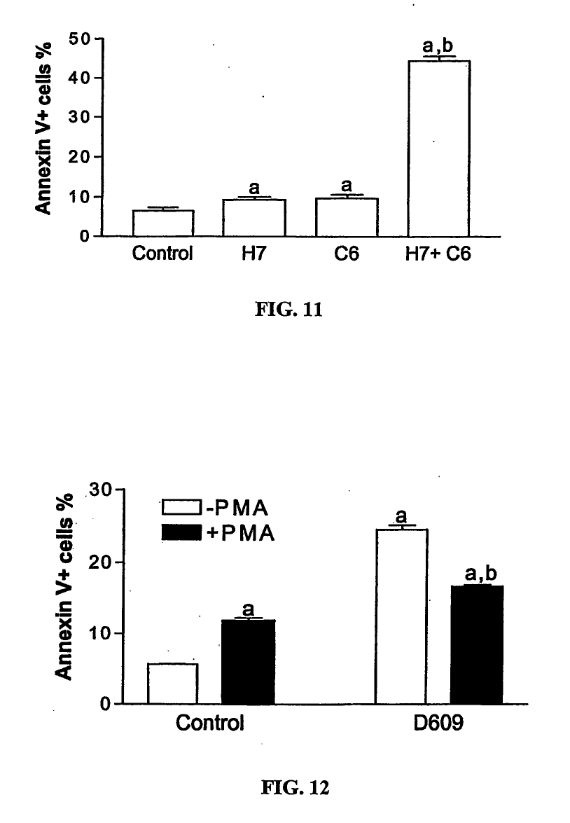

It is well known that ceramide functions as a negative cell regulator that can induce cell cycle arrest, senescence or apoptosis. In contrast, DAG stimulates cell proliferation and promotes cell survival, primarily via activation of PKC. To determine if the changes in the levels of ceramide and DAG resulting from SMS inhibition contribute to D609-induced apoptosis in U937 cells, the cells were incubated with vehicle, H7 (a PKC inhibitor), ceramide, or H7 plus ceramide. U937 cells (1×10⁶/ml) were incubated with 5 μM C6-ceramide and 25 μM H7 alone or combination for 24 hr at 37° C. Apoptosis of the cells was measured by Annexin V-FITC staining and flow cyotmetry and expressed as percent of Annexin V positive cells. a, p<0.05 vs Control; b, p<0.05 vs H7- or C6-treated cells (FIG. 11).

It was found that both H7 and ceramide induced U937 cell apoptosis in a dose-dependent manner. When the cells were incubated with a low dose of H7 (25 μM) or ceramide (5 μM), the induction of apoptosis was modest by either agent (FIG. 11). However, the combined treatment with the same low doses of H7 and ceramide resulted in a synergistic induction of apoptosis (FIG. 11).

Furthermore, when U937 cells were treated with PMA (an activator of PKC) prior to their exposure to D609, PMA partially attenuated D609-induced apoptosis in these cells (FIG. 12). In these experiments, U937 cells were pre-incubated with PMA (25 nM) for 30 min. and then were cultured with vehicle or D609 (150 μM) for 24 hrs. Apoptotic cells were analyzed by Annexin V-FITC staining and flow cytometry and the results are expressed as means±SEM (n=3). a, p<0.001 vs control without PMA; b, p<0.05 vs D609 treatment alone (FIG. 12).

These results indicate that D609-induces tumor cell apoptosis primarily via inhibition of SMS, which results in an increase in the level of ceramide and a decrease in the level of DAG in favor of apoptosis induction.

### Example 6

### Disparity of In Vitro and In Vivo Antitumor Effects of D609

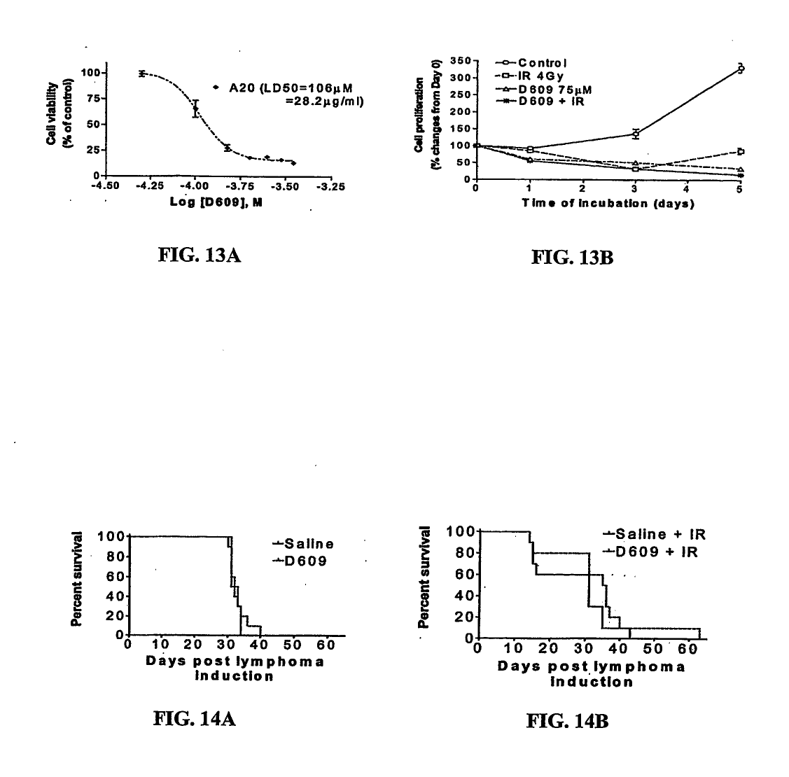

Although D609 is a selective antitumor agent and exhibits potent cytotoxicity against a variety of tuior cells in vitro, it lacks significant therapeutic efficacy against cancer in vivo. Studies were conducted to evaluate the effects of D609 and/or IR on A20 cell viability and growth in vitro. The cell viability was analyzed by MTT assay after A20 cells were cultured with vehicle or different concentrations of D609 for 48 hrs. The results, shown in FIG. 13A, are expressed as percent of vehicle control. A20 cells were untreated (Control), irradiated (IR 4 Gy), incubated with D609 alone, or treated with both D609 and IR. The cell viability and/or proliferation were measured by MTT assay at different times after the cells were placed into well of 96-well plate (2×10⁴/0.2 ml/well). The results are expressed as means±SEM (n=3) (FIG. 13B).

The results show that D609 exhibits high cytotoxicity against A20 cells (a murine B cell lymphoma cells) in vitro. The LD₅₀ value of D609 against A20 cells is about 106 μM or 28.2 μg/ml (FIG. 13A). A20 cells are also sensitive to IR-induced cell death. However, treatment of A20 cells with D609 did not protect the cell from IR, instead, it enhanced their response to IR. Particularly, on day 5 after exposure to IR, a few of the IR resistant cells started to grow back in cultures treated with IR alone, while cell proliferation was further suppressed in cultures treated with D609 alone or in combination with IR (FIG. 13B).

Studies were then conducted to determine whether there were therapeutic effects of D609 and/or IR on A20 lymphoma in vivo. A20 lymphoma was induced in normal BALB/c male mice by the tail vein injection of 5×10⁵ cultured A20 cells. Two days later, the mice were assigned to groups of 10 mice each and received i.v. 1) saline, 2) 50 mg/kg D609, 3) saline plus 6.0 Gy irradiation or 4) D609 plus irradiation. Total body irradiation was given 10 min after D609 injection. Survival of each mouse was then followed by daily monitoring and weighing (5×/week).

However, mice inoculated with A20 cells exhibited no significant therapeutic response to either D609 or IR or treatment with both (FIG. 14A, FIG. 14B). Similar results showing a lack of therapeutic efficacy for D609 have been reported for other types of mouse and human xenograft tumor models, including mouse Lewis lung cancer (Amtmann and Sauer, 1990; Schick et al., 1989; Sauer et al., 1990). These results indicate that the disparity between the in vitro and in vivo antitumor activities of D609 may reflect its poor pharmacokinetics due to the rapid metabolism of D609. This rapid metabolism may result in low levels of D609 reaching the target tumor.

### Example 7

### Rational Design and Synthesis of Prodrug Forms of D609

This example pertains to the synthesis of two series of D609 prodrugs: an S-(alkoxyphosphoryl)- and an S-(alkoxylacyl)-D609 series. The following is a summary of the approaches used to synthesize the phosphoryl analog designated compound 7 in FIG. 4 and the acyl analogs in FIG. 2.

Synthesis of the alkoxyphosphoryl prodrug: The synthesis scheme for the alkoxyphosphoryl prodrug designated compound 7 in FIG. 4 was developed based on the work of Krise et al., 1999, which is specifically herein incorporated by reference, and is shown in the scheme shown in FIG. 4. The (chloromethoxy) di(t-butoxy)phosphoryl was synthesized according to the procedure of Krise et al., 1999, and reacted with the potassium salt of D609 in acetonitrile. The reaction occurred rapidly and generated the desired S-(methyleneoxy) di(t-butoxy)phosphoryl D609 (compound 7) in 65% yield, after purification by silica gel chromatography.

Synthesis of the alkoxyacyl prodrugs (R═CH₃, n-propyl and t-butyl): A series of S-(alkoxylacyl)-D609 prodrugs was rationally designed and synthesized as illustrated in FIG. 2. The syntheses of the alkoxyacyl prodrugs 1, 2 and 3 were developed based on the work of Nudelman et al. (Nudelman et al., 2001). Chloromethyl acetate was synthesized according to the procedure of Bodor et al. (Bodor et al., 1983). The reaction of this alkylating agent with the potassium salt of D609 generated the desired S-methyleneoxyacetyl D609 (1) which was isolated by column chromatography in 64% yield. The synthesis of the desired S-methyleneoxybutyryl D609 (2) and S-methyleneoxypivalolyl D609 (3) was accomplished by the same procedure, beginning with the commercially available chloromethyl butyrate and chloromethyl pivalate, respectively. The yield of 2 and 3 after column chromatography was 73% and 86%, respectively. The purity of these prodrugs was >97% by HPLC analysis and the identity of these compounds was confirmed by ¹H-NMR and ¹³C-NMR spectroscopy.

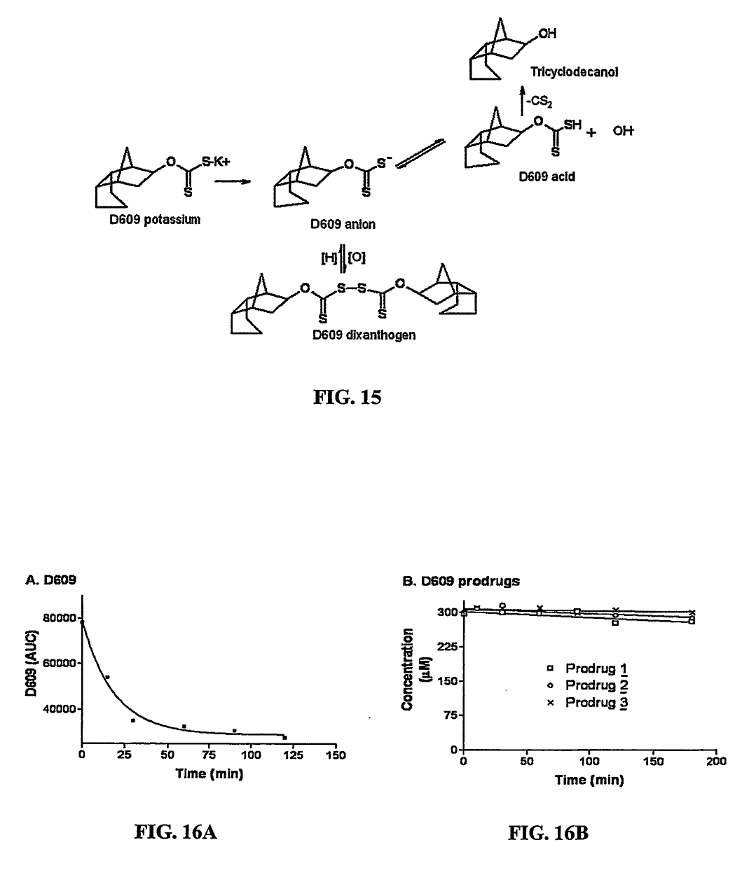

Stability and hydrolytic property of D609 prodrugs: A HPLC assay was initially developed to examine the stability of D609 and D609 prodrugs. The assay showed that D609 rapidly disappeared in saline solution at room temperature (24° C.) with a T_{1/2} about 19.5 min (FIG. 15 and FIG. 16A). The disappearance of D609 in saline is likely due to its oxidation, as the rate of the disappearance was accelerated even further by the addition of low concentrations of mild oxidants, such as H₂O₂ (data not shown). Because of its rapid disappearance after being dissolved in saline, a linear standard curve of D609 could not be constructed using the HPLC analysis and this, the results were expressed as net area under curves (AUC). Compared to D609, D609 prodrugs 1, 2 and 3 are highly stable and their concentrations barely changed during a 3-h incubation in saline, suggesting that no significant spontaneous oxidation and hydrolysis of these compounds occurred (FIG. 16B). Similarly, D609 prodrug S-(methyleneoxy di(t-butoxy)phosphoryl D609 (compound 7 in FIG. 4), when compared to D609, is highly stable. The concentrations of these D609 prodrugs remained steady Even after 48-h incubation in saline (data not shown).

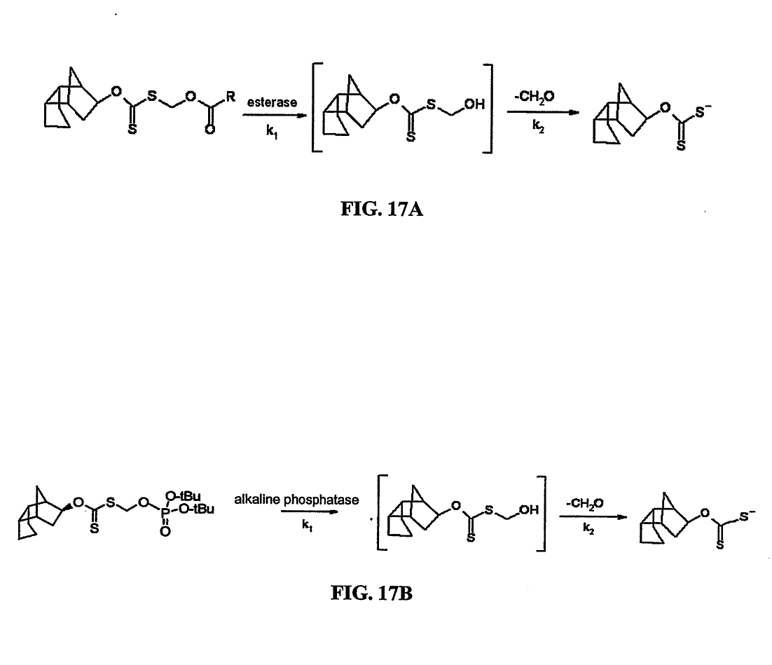

Esterase-catalyzed hydrolysis of D609 prodrugs: The three S-(alkoxylacyl)-D609 prodrugs (i.e. prodrugs 1, 2 and 3 FIG. 2) and the compound 7 (FIG. 4) prodrug are designed to release D609 in two steps: a) phosphatase or esterase-catalyzed hydrolysis of the phosphate ester or acyl ester bond (k₁); followed by b) conversion of the resulting hydroxymethyl D609 to formaldehyde and D609 (k₂) (FIG. 17).

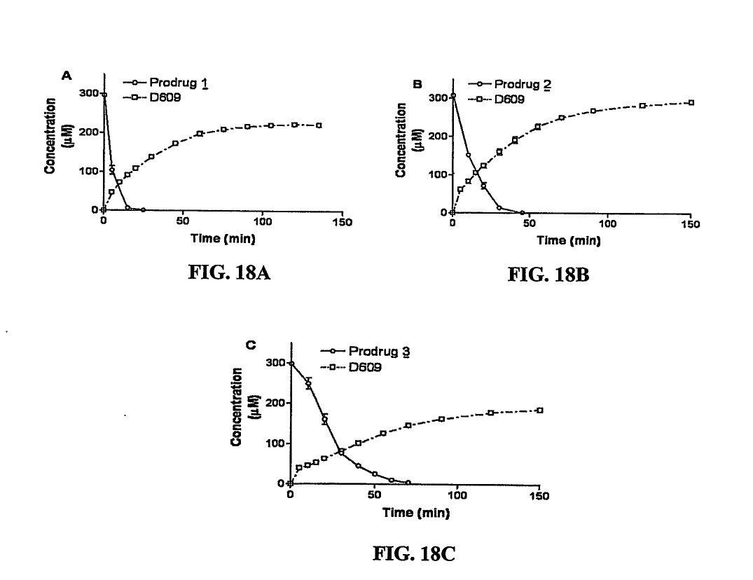

To determine the hydrolytic property of D609 prodrugs 1, 2, and 3 (300 μM in 15% DMSO/PBS, pH7.4), these prodrugs were incubated with 0.1 unit/ml PLE at 37° C. After various times during incubation, the rate of hydrolysis of these D609 prodrugs was monitored by HPLC analysis. The release of D609 was determined by measuring the colorimetric assay of D609 with DTNB, since the concentrations of D609 can be measured more accurately by DTNB than by HPLC due to the rapid oxidation of D609 and the sample manipulations required for the HPLC assay. The concentration of D609 (μM) was calculated based on a linear D609-DTNB standard curve. The pseudo-first-order plots for the hydrolysis of these prodrugs were constructed from the logarithm of remaining ester versus time (FIG. 18). The end points of the reaction (150 min) were defined as when the hydrolysis of these prodrugs was over 99% complete and the release of D609 reached plateau. The pseudo-first-order rate constant (K_{obs}) and T_{1/2} (=0.693/K_{obs}) were calculated based on the slope of the linear portion of the curve for each of these prodrugs and are presented in Table 1 (Gilmer et al., 2002).

TABLE 1
Hydrolysis of D609 prodrugs by esterase

| | k_{obs} | T_{1/2} | |
|---|---|---|---|
| | (min⁻¹) | (min) | % D609 |
| Prodrug 1 | 2.074 × 10⁻¹ | 3.34 | 71 |
| Prodrug 2 | 6.532 × 10⁻² | 10.61 | 93 |
| Prodrug 3 | 2.984 × 10⁻² | 23.22 | 60 |

It was found that prodrug 1 had the shortest T_{1/2}, followed by prodrug 2, and then prodrug 3. This finding indicates that the steric bulkiness of the acyl group (R—) can affect the esterase-catalyzed hydrolysis of the acyl ester bond, as increases in the steric bulk of the acyl group in prodrug 2 and 3 slow the hydrolysis of D609 prodrugs by esterase (FIG. 2 and FIG. 18).

The hydrolysis of these prodrugs resulted in the release of D609. The rate of D609 release was slower than that of prodrug hydrolysis (FIG. 18). This finding indicates that the esterase-catalyzed hydrolysis of the acyl ester bond (k₁) of a D609 prodrug was more facile than the conversion of the resulting hydroxymethyl D609 to formaldehyde and D609 (k₂) (FIG. 16). As shown in FIG. 18, the complete hydrolysis of these prodrugs by esterase led to near-quantitative 60-93% molar recovery of D609. Specifically, the recovery rates of D609 were 71%, 93% and 60% for prodrugs 1, 2, and 3 respectively (FIG. 18 & Table 1). Thus, among these three prodrugs, prodrug 2 gave the highest recovery of D609 after esterase hydrolysis (FIG. 18 & Table 1).

Similarly, compound 7 (FIG. 4) (222 μM) was completely hydrolyzed by alkaline phosphatase (3.125 U/ml, EC 3.1.3.1, from Sigma) in a glycine buffer solution (1 mM ZnCl₂, 1 mM MgCl₂, and 0.1 M glycine, pH 9.0) at 37° C. within 60 min (Krise et al., 1999).

### Example 8

### Hydrolysis of D609 Prodrug 2 in Plasma

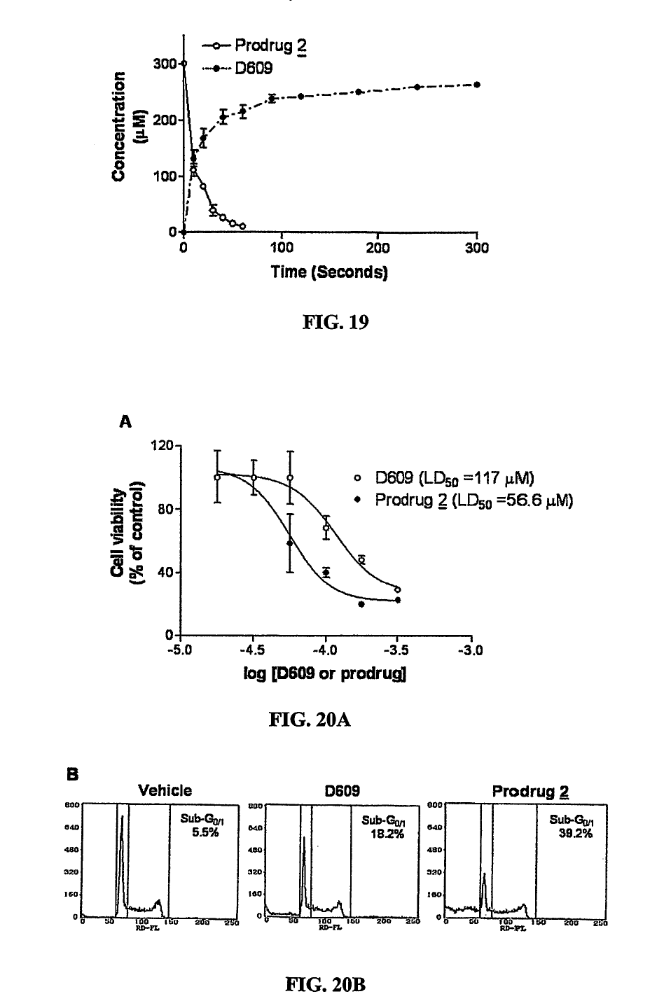

Based on the superior recovery of D609 following hydrolysis by PLE in saline, prodrug 2 was selected for further analysis to determine its hydrolytic property in plasma. For this analysis, prodrug 2 was dissolved in DMSO and then diluted into rat plasma (300 μM in 15% DMSO/plasma). After incubation at 37° C., aliquots (100 μl) of the plasma were removed at various times and immediately mixed with an equal volume of DTNB in acetonitrile (3 mM DTNB). Acetonitrile was used to quickly inactivate plasma esterases and to precipitate plasma proteins. After removal of the precipitated plasma proteins by centrifugation, the concentrations of the D609 prodrug and D609 in the clear plasma supernatants were determined by HPLC and DTNB assays, respectively, as described above. As shown in FIG. 19, prodrug 2 underwent rapid hydrolysis in plasma. The complete hydrolysis of prodrug 2 in plasma was achieved within 60 sec. The K_{obs} and T_{1/2} for prodrug 2 are 9.168×10⁻² sec⁻¹ and 7.559 sec, respectively. Correspondingly, the concentrations of D609 in plasma went up rapidly and reached a plateau in less than 100 sec after the prodrug was added to rat plasma. The complete hydrolysis of prodrug 2 resulted in the release of 88% of D609 based on the initial molar quantity of the prodrug.

### Example 9

### Prodrug Modification Increases D609 Tumor Cytotoxicity

D609 is a selective tumor cytotoxic agent that has the ability to induce tumor cell death by apoptosis (Amtmann and Sauer, 1987; Bettaieb et al., 1999; Meng et al., 2004; Porn-Ares et al., 1997). To determine if prodrug modification increases the biological activity of D609 against tumor, the inventors compared the tumor cell cytotoxicity of prodrug 2 with that of D609 in U937 leukemia cells. As shown in FIG. 20A, incubation of U937 cells with prodrug 2 and D609 resulted in a dose-dependent reduction in cell viability. The decrease in cell viability was associated with an increase in the number of the sub-G_{0/1} cells (FIG. 20B), indicating that both prodrug 2 and D609 are capable of inducing apoptosis in U937 leukemia cells. However, the cells treated with prodrug 2 showed a significantly greater reduction in cell viability and increase in the sub-G_{0/1} cells than D609-treated cells, suggesting that prodrug 2 is more cytotoxic to U937 cells than D609 (FIG. 20). This suggestion is confirmed by the fact that prodrug 2 has a significantly lower LD₅₀ value than that of D609 (56.6 μM vs 117 μM) against U937 cells. Similarly, prodrug 2 also exerted a greater cytotoxicity than D609 against Jurkat T-cell leukemia cells (LD₅₀: prodrug 2 44.26 μM vs D609 63.97 μM) and STM91-01 malignant rhabdoid tumor cells (LD₅₀: prodrug 2 87.10 μM vs D609 545.75 μM). In contrast, prodrug 2 was less toxic to the normal human diploid fibroblasts-WI38 cells than D609 (LD₅₀: prodrug 2 333.43 μM vs D609 267.51 μM). This result indicates that prodrug modification not only increases the cytotoxicity of D609 against tumor cells, but more importantly it also reduces its toxicity to normal cells.

### Example 10

### Prodrug Modification Increases the Inhibitory Effect of D609 on Sphingomyelin Synthase (SMS)

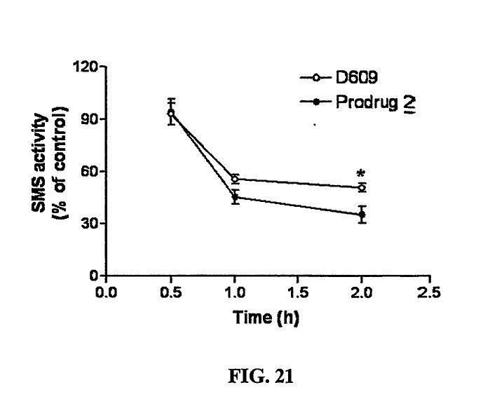

The inventors have recently identified that sphingomyelin synthase (SMS) is a potential molecular target of D609 (Luberto and Hannun, 1998; Meng et al., 2004). Inhibition of SMS activity increases the intracellular level of ceramide and decreases that of diacylglycerol (DAG) in favor of induction of tumor cell apoptosis (Luberto and Hannun, 1998; Meng, et al., 2004). The inventors therefore compared the effect of prodrug 2 with that of D609 on SMS in U937 cells. The study showed that the enzymatic activity of SMS in U937 cell lysates was linear with the amount of protein and time of the reaction (data not shown). Based on this assay, the optimal conditions of the assay were selected. Under these conditions, incubation of U937 cells with D609 or prodrug 2 (177 μM) resulted in a time-dependent inhibition of SMS activity (FIG. 21). However, the inhibition was significantly greater in prodrug 2-treated cells than that of D609-treated cells (p<0.05), demonstrating that prodrug modification significantly increased the inhibitory effect of D609 on SMS.

### Example 11

### Prodrug Modification Augments D609-Induced Increase in Ceramide

The effects of D609 and prodrug 2 on the level of ceramide in U937 cells were also examined since D609 can increase the level of ceramide via inhibition of SMS and stimulation of the de novo synthesis of ceramide (Luberto and Hannun, 1998; Meng et al., 2004; Perry and Ridgway, 2004). An ESI/MS/MS analysis was used to profile the changes in the levels of various species of ceramide in U937 cells after they were incubated with 177 μM D609 or prodrug 2. The cells treated with D609 or prodrug 2 showed a significant increase in almost all species of ceramide, except that the level of C₂₋₄-ceramide was not changed in the cells treated with D609 and that of C_{18:1}-ceramide was below detection limits for all cells examined (Table 2 and data not shown). Cells treated with prodrug 2 exhibited a greater increase in the levels of various species of ceramide than these treated with D609. U937 cells treated with D609 exhibited an about 1.61-fold increase in the level of total ceramide compared to that of vehicle-treated cells p<0.001). The increase (1.84-fold) was significantly greater in the cells treated with prodrug 2 than that of D609-treated cells p<0.05).

TABLE 2
D609- and Prodrug 2-induced changes in ceramide profiles in U937 cells*

| Treatment | C₁₄-Cer | dhC₁₆-Cer | C₁₆-Cer | C₁₈-Cer | C₂₀-Cer | C_{24:1}-Cer | C₂₄-Cer | Total-Cer |
|---|---|---|---|---|---|---|---|---|
| Vehicle | 0.019 | 0.403 | 1.027 | 0.057 | 0.007 | 0.494 | 0.357 | 2.363 |
| | (0.004) | (0.009) | (0.032) | (0.006) | (0.001) | (0.040) | (0.012) | (0.012) |
| D609 | 0.038^{a} | 0.832^{a} | 1.612^{a} | 0.213^{a} | 0.036^{a} | 0.669^{a} | 0.416 | 3.816^{a} |
| | (0.004) | (0.025) | (0.046) | (0.023) | (0.005) | (0.077) | (0.060) | (0.197) |
| Prodrug 2 | 0.054^{a,b} | 0.951^{a,b} | 1.713^{a} | 0.262^{a} | 0.031^{a} | 0.863^{a,b} | 0.482^{a} | 4.358^{a,b} |
| | (0.008) | (0.042) | (0.134) | (0.044) | (0.002) | (0.037) | (0.037) | (0.222) |

*U937 cells were incubated with vehicle (0.5% DMSO) or 177 □M D609 or Prodrug 2 for 4 h. Total lipids were extracted and analyzed for ceramide by ESI/MS/MS. The values are expressed as pmole ceramide/nmole lipid Pi and presented as mean and SD (in parenthesis) (n = 3). Cer, Ceramide; dh-Cer, Dihydroceramide
^{a}p < 0.05 to 0.001 vs vehicle.
^{b}p < 0.05 to 0.001 vs D609.

### Example 12

### Discussion

D609 is a member of a new class of nucleophilic sulfur pharmaceutical agents that contains a xanthate group (C(═S)S⁻/—C(═S)SH). Similar to the —SH group of WR1065, the xanthate group of D609 can be easily oxidized to form a disulfide bond (Giron-Calle et al., 2002; Rao, 1971; Zhou et al., 2001). This oxidative instability may contribute to its poor antitumor activity in vivo (Amtrnann and Sauer, 1990; Sauer, et al., 1990; Schick et al., 1989b).

Three S-(alkoxyacyl) D609 prodrugs were synthesized by varying the steric bulkiness of the acyl group. They are S-methyleneoxyacetyl D609 (1), S-methyleneoxybutyryl D609 (2) and S-methyleneoxypivalyl D609 (3). These prodrugs have increased chemical stability, as no significant hydrolysis and oxidation of these compounds was observed after they were dissolved in saline and kept at room temperature for up to 48 h. However, when they were incubated with 0.1 unit/ml PLE at 37° C. they were readily hydrolyzed with a T_{1/2} value ranges from 3.34 to 23.22 minutes. Among these prodrugs, prodrug 1 had the shortest T_{1/2}, which was followed by prodrug 2 and then by prodrug 3, indicating that an increase in the steric bulkiness of the acyl group hinders the esterase-catalyzed hydrolysis of the acyl ester bond. Further modification of the S-(alkoxyacyl) group allows tailoring of the hydrolysis rates and pharmacokinetic parameters of the D609 prodrugs. In addition, the S-(alkoxyacyl) modification strategy can be applicable to the development of other redox active sulfur compounds to produce ester prodrugs. These ester prodrugs should have better drug absorption and distribution properties than phosphorothioate-modified sulfur pro drugs, because phosphorothioate-modified prodrugs, such as amifostine, exist as an ionized phosphorothioic acidic molecule at a physiological pH (7.4), which contributes to their poor distribution and rapid clearance in urine (Culy and Spencer, 2001; Poggi et al., 2001; pencer and Goa, 1995; Capizzi, 1999).

The hydrolysis of S-(alkoxyacyl) D609 prodrugs by esterase produces a hydroxymethyl-D609 intermediate that spontaneously breaks down to release the parent drug (D609) and formaldehyde. The complete hydrolysis of prodrugs 1, 2, and 3 resulted in approximately 71%, 93% and 60% molar recovery of D609, respectively. Hydrolysis of prodrug 2, and the subsequent release of D609, was much faster with rat plasma than with PLE (0.1 unit/ml). The shorter half-life of prodrug 2 in rat plasma may be due to higher levels of esterases and/or to a greater susceptibility of this prodrug to the esterases present in rat plasma. At any rate, the hydrolysis of prodrug 2 in rat plasma resulted in the almost complete (88%) release of D609.

It was found that in in vitro assays prodrug 2 was biologically more active against various human tumor cell lines but less toxic to normal human diploid fibroblasts than D609. This result shows that the S-(alkoxyacyl) prodrug modification significantly improves the antitumor activity of D609, in part, by increasing the chemical stability of D609. For example, it was shown that prodrug 2 has a greater apparent potency than D609 in induction of apoptosis in U937 cells and had a significantly lower LD₅₀ value than that of D609 (56.6 μM vs117 μM). In addition, the increased tumor cell cytotoxicity of prodrug 2 was associated with an augmented inhibition of SMS, a potential molecular target of D609, resulting in a greater elevation in the ceramide levels in U937 cells. It has been suggested that D609 may selectively kill tumor cells by elevating the level of ceramide via direct inhibition of SMS. Therefore, these observations suggest that the tumor cell cytotoxicity of the S-(alkoxyacyl) prodrug is likely mediated by D609 released from the prodrug after its hydrolysis, since the prodrug induces tumor cell death by affecting the same molecular target and pathway.

All of the compounds, compositions, and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Amtmann and Sauer, Cancer Lett., 35:237-244, 1987.
Amtmann and Sauer, Int. J. Cancer, 45:1113-1118, 1990.
Amtmann et al., Exp. Cell Res., 161:541-550, 1985.
Amtmann et al., J. Gen. Virol., 66(Pt 9):1953-1960, 1985.
Amtmann, Drugs Exp. Clin. Res., 22:287-294, 1996.
Bai et al., J. Pharmacol. Exp. Ther., 309 (3):1051-1059, 2004.
Bettaieb et al., Mol. Pharmacol., 55:118-125, 1999.
Bettaieb et al., Mol. Pharmacol., 55:118-125, 1999.
Bodor et al., J. Organic Chemistry, 48:5280-5284, 1983.
Capizzi, Semin. Oncol., 26:3-21, 1999.
Chevion et al., Free Radic. Biol. Med., 22:411-421, 1997.
Culy and Spencer, Drugs, 61:641-684, 2001.
Druker and Lydon, J. Clin. Invest., 105:3-7, 2000.
Enomoto et al., Cancer Lett., 155:137-144, 2000.
Furstenberger et al., Int. J. Cancer, 43:508-512, 1989.
Gazdar and Minna, Proc. Natl. Acad. Sci. USA, 98:10028-10030, 2001.
Gibbs, Science, 287:1969-1973, 2000.
Gilmer et al., Eur. J. Pharm. Sci., 16:297-304, 2002.
Giron-Calle et al., J. Pharmacol. Exp. Ther., 301:87-94, 2002.
Greenberger et al., Antioxid. Redox. Signal, 3:347-359, 2001.
Hansen et al., J. Immunol. Methods, 119:203-210, 1989.
Hospers et al., Br. J. Cancer, 80:629-638, 1999.
Krise et al., J. Pharm. Sci., 88:928-932, 1999a.
Krise et al., J. Pharm. Sci., 88:922-927, 1999b.
Lauderback et al., Free Radic. Res., 37:355-365, 2003.
Li et al., Proc. Natl. Acad. Sci. USA, 95:7748-7775, 1998.
Luberto and Hannun, J. Biol. Chem., 273:14550-14559, 1998.
Luberto et al., J. Biol. Chem., 275:14760-14766, 2000.
Machleidt et al., J. Exp. Med., 184:725-733, 1996.
Meng et al., Exp. Cell Res., 292:385-392, 2004.
Nudelman et al., Eur. J. Med. Chem., 36:63-74, 2001.
Paris et al., Science, 293:293-297, 2001.
Perry and Ridgway, J. Lipid Res., 45:164-73, 2004.
Pettus et al., FASEB J., 17:1411-1421, 2003b.
Pettus et al., Rapid Commun. Mass Spectrom., 17:1203-1211, 2003a.
Poggi et al., Curr. Probl. Cancer, 25:334-411, 2001.
Porn-Ares et al., Exp. Cell Res., 235:48-54, 1997.
Rao, In: Xanthlates and related compounds. Marcel Dekker, Inc., New York, 1971.
Rasmussen and Leonard, J. Amer. Chem. Soc., 89(21), 5439-5445, 1967.
Remington's Pharmaceutical Sciences, 15^{th} ed., 33:624-652, Mack Publishing Company, Easton, Pa., 1980.
Riboni et al., J. Biol. Chem., 276:12797-12804, 2001.
Santana et al., Cell, 86:189-199, 1996.
Sauer et al., Cancer Lett., 53:97-102, 1990.
Schick et al., Cancer Lett., 46:143-147, 1989a.
Schick et al., Cancer Lett., 46:149-152, 1989b.
Schutze et al., Cell, 71:765-776, 1992.
Schutze et al., J. Exp. Med., 174:975-988, 1991.
Sinkula and Yalkowsky, J. Pharm. Sci., 64:181-210, 1975.
Smith and Clark, In: Textbook of organic medicine and pharmaceutical chemistry, Delgado and Remers, (Eds.), 123-138, Lippincott-Raven, Philadelphia, 1998.
Smith and Rutledge, Natl. Cancer Inst. Monogr., 42:141-143, 1975.
Spencer and Goa, Drugs, 50:1001-1031, 1995.
Wang et al., Bioorg. Med. Chem., 6:417-426, 1998.
Young et al., N. Engl. J. Med., 7:299(23):1261-1266, 1978.
Zhou et al., Int. J. Radiat. Biol., 77:763-772, 2001.
Zhou et al., J. Pharmacol. Exp. Ther., 298:103-109, 2001.

1. A compound of formula (I) 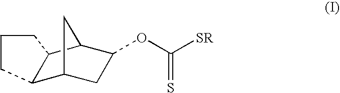
wherein R is a heteroatom substituted alkyl moiety;
or a pharmaceutically acceptable salt thereof.

2. The compound of claim 1, wherein R is an alkyl or alkoxy moiety.

3. The compound of claim 2, wherein R is an alkoxyphosphoryl or alkoxyacyl moiety.

4. The compound of claim 3, wherein the compound of formula (I) is: 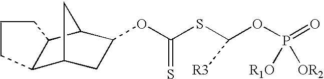
wherein R¹, R², and R³ are independently H or alkyl moieties;
or a pharmaceutically acceptable salt thereof.

5. A compound of claim 3, wherein the compound of formula (I) is: 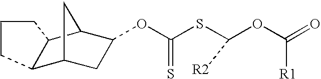
wherein R¹ and R² are independently H or alkyl moieties;
or a pharmaceutically acceptable salt thereof.

6. A pharmaceutical composition comprising a compound of claim 1 and a pharmaceutically acceptable excipient.

7. A method of treating a disease or disorder in a subject, comprising:
a) obtaining a composition comprising a compound of formula (I): 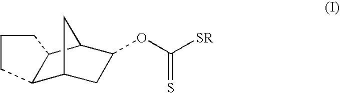
wherein R is a heteroatom substituted alkyl moiety;
or a pharmaceutically acceptable salt thereof; and
b) administering a therapeutically effective amount of the composition to the subject.

8. The method of claim 7, wherein R is an alkyl or alkoxy moiety.

9. The method of claim 8, wherein R is an alkoxyphosphoryl or alkoxyacyl moiety.

10. The method of claim 9, wherein the compound of formula (I) is: 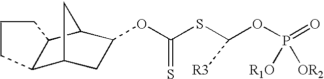
wherein R¹, R² and R³ are independently H or alkyl moieties;
or a pharmaceutically acceptable salt thereof.

11. The method of claim 9, wherein the compound of formula (I) is: 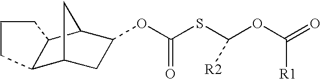
wherein R¹ and R² are independently H or alkyl moieties;
or a pharmaceutically acceptable salt thereof.

12. The method of claim 7, wherein the subject is a mammal.

13. The method of claim 12, wherein the mammal is a human.

14. The method of claim 7, wherein the disease is an autoimmune disease, and inflammatory disease, a neurodegenerative disease, a disease associated with ischemia and reperfusion injury, trauma, atherosclerosis, ageing, cancer, viral infection, UV-induced radiation injury, or ionizing radiation-induced injury.

15. The method of claim 14, wherein the autoimmune disease is systemic lupus, chronic thyroiditis, Graves disease, autoimmune gastritis, autoimmune hemolytic anemia, autoimmune neutropenia, or thrombocytopenia.

16. The method of claim 14, wherein the inflammatory disease is rheumatoid arthritis, organ transplant rejection, graft versus host disease, endotoxemia, sepsis, septic shock, uveitis, inflammatory peritonitis, or pancreatitis.

17. The method of claim 14, wherein the neurodegenerative disease is Alzheimer disease, Parkinson's disease, Huntington's disease, Kennedy's disease, prion disease, multiple sclerosis, amyotrophic lateral sclerosis, or spinal muscular atrophy.

18. The method of claim 14, wherein the disease associated with ischemia and reperfusion injury is a stroke or myocardial infarction.

19. The method of claim 14, wherein the cancer is breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, leukemia, stomach cancer, pancreatic cancer, testicular cancer lymphoma, or multiple myeloma.

20. The method of claim 14, wherein the trauma is traumatic brain injury spinal cord injury, or burn injury.

21. The method of claim 7, wherein the disease or disorder is a disease or disorder associated with oxidative stress.

22. The method of claim 7, wherein administration of the composition comprises oral administration, intravenous administration, intraarterial administration, topical administration, intratumoral administration, regional administration, intrathecal administration, intraperitoneal administration, intraocular administration, or inhalational administration.

23. A method of protecting normal tissue in a subject from the toxicity associated with treatment of a disease with ionizing radiation or a chemotherapeutic agent, comprising:
a) obtaining a composition comprising a compound of formula (I): 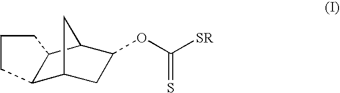
wherein R is a heteroatom substituted alkyl moiety;
or a pharmaceutically acceptable salt thereof; and
b) concurrently or consecutively administering to the subject a prophylactically effective amount of the composition and the ionizing radiation or chemotherapeutic agent.

24. The method of claim 23, wherein R is an alkyl or alkoxy moiety.

25. The method of claim 24, wherein R is an alkoxyphosphoryl or alkoxyacyl moiety.

26. The method of claim 25, wherein the compound of formula (I) is: 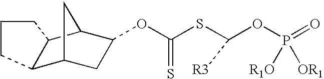
wherein R¹, R² and R³ are independently H or alkyl moieties;
or a pharmaceutically acceptable salt thereof.

27. The method of claim 25, wherein the compound of formula (I) is: 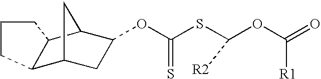
wherein R¹ and R² are independently H or an alkyl moieties;
or a pharmaceutically acceptable salt thereof.

28. The method of claim 23, wherein the subject is a mammal.

29. The method of claim 28, wherein the mammal is a human.

30. The method of claim 23, wherein the disease is an autoimmune disease, and inflammatory disease, a neurodegenerative disease, a disease associated with ischemia and reperfusion injury, trauma, atherosclerosis, ageing, cancer, or a viral infection.

31. The method of claim 30, wherein the disease is cancer.

32. The method of claim 31, wherein the cancer is breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, leukemia, stomach cancer, pancreatic cancer, testicular cancer lymphoma, or multiple myeloma.

33. The method of claim 23, wherein the chemotherapeutic agent is doxorubicin, daunorubicin, methotrexate, tamoxifen, paclitaxel, topotecan, LHRH, mitomycin C, etoposide tomudex, podophyllotoxin, mitoxantrone, colchicine, endostatin, fludarabin, mitomycin, actinomycin D, bleomycin, cisplatin, VP16, an enedyine, taxol, vincristine, vinblastine, carmustine, melphalan, cyclophosphamide, chlorambucil, busulfan, lomustine, 5-fluorouracil, gemcitabine, BCNU, or camptothecin.

34. The method of claim 23, wherein administering a prophylactically effective amount of the composition comprises oral administration, intravenous administration, intraarterial administration, topical administration, local administration into a tumor, intrathecal administration, intraperitoneal administration, intraocular administration, or inhalational administration.

35. The method of claim 23, wherein the prophylactically effective amount of the composition and the ionizing radiation or chemotherapeutic agent are concurrently administered.

36. The method of claim 23, wherein the prophylactically effective amount of the composition and the ionizing radiation or chemotherapeutic agent are consecutively administered.

37. A method of treating a disease or disorder in a subject, comprising:
a) obtaining a composition comprising a compound of formula (I): 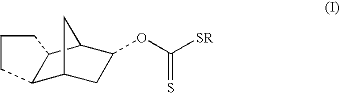
wherein R is a heteroatom substituted alkyl moiety;
or a pharmaceutically acceptable salt thereof; and
b) concurrently or consecutively administering a therapeutically effective amount of the composition and ionizing radiation or a chemotherapeutic agent to the subject.

38. The method of claim 37, wherein R is an alkyl or alkoxy moiety.

39. The method of claim 38, wherein R is an alkoxyphosphoryl or alkoxyacyl moiety.

40. The method of claim 39, wherein the compound of formula (I) is: 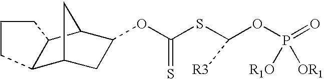
wherein R¹, R², and R³ are independently H or alkyl moieties;
or a pharmaceutically acceptable salt thereof.

41. The method of claim 39, wherein the compound of formula (I) is: 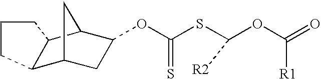
wherein R¹ and R² are independently H or alkyl moieties;
or a pharmaceutically acceptable salt thereof.

42. The method of claim 37, wherein the subject is a mammal.

43. The method of claim 42, wherein the mammal is a human.

44. The method of claim 37, wherein the disease is cancer.

45. The method of claim 44, wherein the cancer is breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, leukemia, stomach cancer, pancreatic cancer, testicular cancer lymphoma, or multiple myeloma.

46. The method of claim 37, wherein the therapeutically effective amount of the composition and the ionizing radiation or chemotherapeutic agent are concurrently administered.

47. The method of claim 37, wherein the therapeutically effective amount of the composition and the ionizing radiation or chemotherapeutic agent are consecutively administered.
